SEQUENCE LISTING (71 sequences)

SEQ ID NO: 1 - DNA, 18 bp, Artificial sequence
  Synthetic sequence
cctcctgctactgctgac

SEQ ID NO: 2 - DNA, 19 bp, Artificial sequence
  Synthetic sequence
gtcagcacaaagtcctctc

SEQ ID NO: 3 - DNA, 22 bp, Artificial sequence
  Synthetic sequence
ctccagaaggccctcagactac

SEQ ID NO: 4 - DNA, 18 bp, Artificial sequence
  Synthetic sequence
gggtcttcattgcggtgg

SEQ ID NO: 5 - DNA, 20 bp, Artificial sequence
  Synthetic sequence
cgtccctcagtcaagaggag

SEQ ID NO: 6 - DNA, 20 bp, Artificial sequence
  Synthetic sequence
gtccctagaagtgcccaaca

SEQ ID NO: 7 - protein, 19 aa, Artificial sequence
  Synthetic sequence
ASNLGLEDIIRKALMGSFD

SEQ ID NO: 8 - protein, 8 aa, Artificial sequence
  Synthetic sequence
TEIEHLVQ

SEQ ID NO: 9 - protein, 11 aa, Artificial sequence
  Synthetic sequence
TEIEHLVQSVC

SEQ ID NO: 10 - protein, 13 aa, Artificial sequence
  Synthetic sequence
TEIEHLVQSVCKS

SEQ ID NO: 11 - protein, 19 aa, Artificial sequence
  Synthetic sequence
TEIEHLVQSVCKSYRETCQ

SEQ ID NO: 12 - protein, 6 aa, Artificial sequence
  Synthetic sequence
LRLEDL

SEQ ID NO: 13 - protein, 6 aa, Artificial sequence
  Synthetic sequence
RLEDLL

SEQ ID NO: 14 - protein, 16 aa, Artificial sequence
  Synthetic sequence
RLEDLLRQRSNIFSRE

SEQ ID NO: 15 - protein, 13 aa, Artificial sequence
  Synthetic sequence
EVTGYQRKSMWEM

SEQ ID NO: 16 - protein, 13 aa, Artificial sequence
  Synthetic sequence
WERCAHHLTEAIQ

SEQ ID NO: 17 - protein, 14 aa, Artificial sequence
  Synthetic sequence
WERCAHHLTEAIQY

SEQ ID NO: 18 - protein, 11 aa, Artificial sequence
  Synthetic sequence
VVEFAKRLSGF

SEQ ID NO: 19 - protein, 8 aa, Artificial sequence
  Synthetic sequence
FAKRLSGF

SEQ ID NO: 20 - protein, 7 aa, Artificial sequence
  Synthetic sequence
AKRLSGF

SEQ ID NO: 21 - protein, 8 aa, Artificial sequence
  Synthetic sequence
CQNDQIVL

SEQ ID NO: 22 - protein, 15 aa, Artificial sequence
  Synthetic sequence
VRMCRAYNADNRTVF

SEQ ID NO: 23 - protein, 12 aa, Artificial sequence
  Synthetic sequence
CRAYNADNRTVF

SEQ ID NO: 24 - protein, 10 aa, Artificial sequence
  Synthetic sequence
FEGKYGGMEL

SEQ ID NO: 25 - protein, 8 aa, Artificial sequence
  Synthetic sequence
FRALGCSE

SEQ ID NO: 26 - protein, 15 aa, Artificial sequence
  Synthetic sequence
LISSIFDFSHSLSAL

SEQ ID NO: 27 - protein, 6 aa, Artificial sequence
  Synthetic sequence
ISSIFD

SEQ ID NO: 28 - protein, 11 aa, Artificial sequence
  Synthetic sequence
ISSIFDFSHSL

SEQ ID NO: 29 - protein, 14 aa, Artificial sequence
  Synthetic sequence
ISSIFDFSHSLSAL

SEQ ID NO: 30 - protein, 9 aa, Artificial sequence
  Synthetic sequence
DFSHSLSAL

SEQ ID NO: 31 - protein, 8 aa, Artificial sequence
  Synthetic sequence
FSHSLSAL

SEQ ID NO: 32 - protein, 9 aa, Artificial sequence
  Synthetic sequence
HFSEDEIAL

SEQ ID NO: 33 - protein, 16 aa, Artificial sequence
  Synthetic sequence
LAFHHHLCKTHRQSIL

SEQ ID NO: 34 - protein, 15 aa, Artificial sequence
  Synthetic sequence
AKLPPKGKLRSLCSQ

SEQ ID NO: 35 - protein, 17 aa, Artificial sequence
  Synthetic sequence
HVERLQIFQHLHPIVVQ

SEQ ID NO: 36 - protein, 12 aa, Artificial sequence
  Synthetic sequence
QIFQHLHPIVVQ

SEQ ID NO: 37 - protein, 10 aa, Artificial sequence
  Synthetic sequence
AAFPPLYKEL

SEQ ID NO: 38 - protein, 11 aa, Artificial sequence
  Synthetic sequence
AAFPPLYKELF

SEQ ID NO: 39 - protein, 8 aa, Artificial sequence
  Synthetic sequence
TEIEHLVQ

SEQ ID NO: 40 - protein, 11 aa, Artificial sequence
  Synthetic sequence
TEIEHLVQSVC

SEQ ID NO: 41 - protein, 13 aa, Artificial sequence
  Synthetic sequence
TEIEHLVQSVCKS

SEQ ID NO: 42 - protein, 19 aa, Artificial sequence
  Synthetic sequence
TEIEHLVQSVCKSYRETCQ

SEQ ID NO: 43 - protein, 19 aa, Artificial sequence
  Synthetic sequence
TEIEHLVQSVCKSYRETCQ

SEQ ID NO: 44 - protein, 20 aa, Artificial sequence
  Synthetic sequence
TEIEHLVQSVCKSYRETCQL

SEQ ID NO: 45 - protein, 5 aa, Artificial sequence
  Synthetic sequence
RLEDL

SEQ ID NO: 46 - protein, 6 aa, Artificial sequence
  Synthetic sequence
RLEDLL

SEQ ID NO: 47 - protein, 16 aa, Artificial sequence
  Synthetic sequence
RLEDLLRQRSNIFSRE

SEQ ID NO: 48 - protein, 13 aa, Artificial sequence
  Synthetic sequence
EVTGYQRKSMWEM

SEQ ID NO: 49 - protein, 13 aa, Artificial sequence
  Synthetic sequence
WERCAHHLTEAIQ

SEQ ID NO: 50 - protein, 14 aa, Artificial sequence
  Synthetic sequence
WERCAHHLTEAIQY

SEQ ID NO: 51 - protein, 11 aa, Artificial sequence
  Synthetic sequence
VVEFAKRLSGF

SEQ ID NO: 52 - protein, 8 aa, Artificial sequence
  Synthetic sequence
FAKRLSGF

SEQ ID NO: 53 - protein, 8 aa, Artificial sequence
  Synthetic sequence
CQNDQIVL

SEQ ID NO: 54 - protein, 15 aa, Artificial sequence
  Synthetic sequence
VRMCRAYNADNRTVF

SEQ ID NO: 55 - protein, 12 aa, Artificial sequence
  Synthetic sequence
CRAYNADNRTVF

SEQ ID NO: 56 - protein, 9 aa, Artificial sequence
  Synthetic sequence
YNADNRTVF

SEQ ID NO: 57 - protein, 10 aa, Artificial sequence
  Synthetic sequence
FEGKYGGMEL

SEQ ID NO: 58 - protein, 9 aa, Artificial sequence
  Synthetic sequence
FRALGCSEL

SEQ ID NO: 59 - protein, 15 aa, Artificial sequence
  Synthetic sequence
LISSIFDFSHSLSAL

SEQ ID NO: 60 - protein, 6 aa, Artificial sequence
  Synthetic sequence
ISSIFD

SEQ ID NO: 61 - protein, 14 aa, Artificial sequence
  Synthetic sequence
ISSIFDFSHSLSAL

SEQ ID NO: 62 - protein, 11 aa, Artificial sequence
  Synthetic sequence
IFDFSHSLSAL

SEQ ID NO: 63 - protein, 9 aa, Artificial sequence
  Synthetic sequence
DFSHSLSAL

SEQ ID NO: 64 - protein, 8 aa, Artificial sequence
  Synthetic sequence
FSHSLSAL

SEQ ID NO: 65 - protein, 9 aa, Artificial sequence
  Synthetic sequence
HFSEDEIAL

SEQ ID NO: 66 - protein, 16 aa, Artificial sequence
  Synthetic sequence
LAFHHHLCKTHRQSIL

SEQ ID NO: 67 - protein, 15 aa, Artificial sequence
  Synthetic sequence
AKLPPKGKLRSLCSQ

SEQ ID NO: 68 - protein, 17 aa, Artificial sequence
  Synthetic sequence
HVERLQIFQHLHPIVVQ

SEQ ID NO: 69 - protein, 12 aa, Artificial sequence
  Synthetic sequence
QIFQHLHPIVVQ

SEQ ID NO: 70 - protein, 10 aa, Artificial sequence
  Synthetic sequence
AAFPPLYKEL

SEQ ID NO: 71 - protein, 11 aa, Artificial sequence
  Synthetic sequence
AAFPPLYKELF

(19) United States
(12) Patent Application Publication
(10) Pub. No.: US 2024/0245633 A1
(43) Pub. Date: Jul. 25, 2024

(54) ROR Gamma Agonists as Enhancers of Protective Immunity

(71) Applicant: University of Florida Research Foundation, Incorporated, Gainesville, FL (US)

(72) Inventors: Patrick Griffin, Jupiter, FL (US); Theodore Kamenecka, Palm Beach Gardens, FL (US); Mi Ra Chang, Jupiter, FL (US); Christelle Doebelin, Jupiter, FL (US)

(21) Appl. No.: 15/768,888

(22) PCT Filed: Nov. 1, 2016
(86) PCT No.: PCT/US16/59841

§ 371 Date: Apr. 17, 2018

Related U.S. Application Data
(60) Provisional application No. 62/250,672, filed on Nov. 4, 2015.

(51) Int. Cl. A61K 31/167; A61K 31/137; A61K 31/18; A61K 31/192; A61K 31/277; A61K 31/341; A61K 31/381; A61K 31/4402; A61K 31/4406; A61K 31/4409

(52) U.S. Cl. CPC A61K 31/167; A61K 31/137; A61K 31/18; A61K 31/192; A61K 31/277; A61K 31/341; A61K 31/381; A61K 31/4402; A61K 31/4406; A61K 31/4409

(57) ABSTRACT

The T cell specific RORgamma isoform RORgammat has been shown to be the key lineage-defining transcription factor to initiate the differentiation program of T_{H}17 and T_{C}17 cells, cells that have demonstrated anti-tumor efficacy, RORgammat controls gene networks that enhance immunity including increased IL17 production and decreased immune suppression. Agonists of RORgammat have been shown to increase the basal activity of RORgammat enhancing T_{H}17 cell proliferation. Here we show that activation of RORgammat using synthetic agonists drives proliferation of cells while decreasing levels of the immune checkpoint protein PD-1, a mechanism that should enhance anti-tumor immunity while blunting tumor associated adaptive immune resistance. Interestingly, putative endogenous agonists drive proliferation of T_{H}17 cells but do not repress PD-1. These findings suggest that synthetic agonists of RORgammat should activate T_{C}17/T_{H}17 cells, decrease the population of Tregs, repress PD-1, and produce IL17 in situ, an immune factor associated with good prognosis in cancer.

### CROSS-REFERENCE TO RELATED APPLICATION

Reference is made to PCT application PCT/US2011/028320, published as WO 2011/115892, “MODULATORS OF THE RETINOIC ACID RECEPTOR-RELATED ORPHAN RECEPTORS”, which is incorporated herein by reference in its entirety. This application claims the priority of U.S. provisional application Ser. No. 62/250,672, filed Nov. 4, 2015, the disclosure of which is incorporated by reference herein in its entirety.

### STATEMENT OF GOVERNMENT SUPPORT

This invention was made with government support under MH108173 awarded by the National Institutes of Health. The government has certain rights in the invention.

### BACKGROUND

The nuclear receptor (NR) superfamily of transcription factors has proven to be rich source of targets for development of therapeutics for a myriad of human diseases. In addition to control by cellular localization and PTM status, the transcriptional activity of most NRs can be modulated (activated or repressed) by small lipophilic molecules such as hormones, vitamins, steroids, oxysterols, retinoids, fatty acids, and synthetic molecules¹. The NR1F subfamily of NRs contains the retinoic acid receptor-related orphan receptors (RORs) that include RORα, RORβ, and RORγ. These receptors have been shown to regulate a wide range of physiological processes, have been implicated in the pathophysiology of disease, and their basal activity can be modulated by sterols²⁻⁴.

The T cell specific isoform of RORγ, known as RORγt, is expressed in thymocytes and regulates survival of T cells during differentiation⁵ and drives the activation and differentiation of CD4⁺ and CD8⁺ cells into IL17-producing helper T cells (T_{H}17) and cytotoxic T cells (Tc17)⁶. T_{H}17 and Tc17 are effector cells that promote inflammation, adaptive immunity and autoimmunity by producing IL17 and other inflammatory cytokines such as IL21. Since T_{H}17 cells do not express granzyme B or perforin and do not appear to have a direct effect on cancer cell proliferation and apoptosis, it is thought that these cells may not mediate direct cytotoxic activity against tumors^{7,8}.

The programmed cell death 1 receptor PD-1 can inhibit T cell activation when bound by the ligand PD-L1. Tumor expression of PD-L1 leads to an inactivation of a T cell immune response to the cancer cells. Activated T cells produce interferon and stimulate PD-L1 on tumor cells and the PD-1/PD-L1 interaction triggers a process that shuts down the immune response reducing proliferation of these effector cells. In the tumor microenvironment, T cells overexpress PD-1 and act in concert to blunt T cell antitumor effects^{9,10}. Among the most promising approaches to activating therapeutic antitumor immunity is the blockade of immune checkpoints. While T_{H}17 cells have a well described role in autoimmune disease, recent evidence suggests that this subset of effector T cells may play a role in immunotherapy if the PD-1 pathway is inactivated¹¹. Enhanced immunity through T-cell activation and blockage of immune checkpoints has transformed cancer treatment with therapies targeting PD-1 showing unprecedented rates of durable clinical responses in patients with various cancers¹²⁻¹⁶.

Several reports have described RORγt synthetic agonists including SR1078, a compound that induced the expression of the ROR target genes FGF21 and G6Pase in cells and in vivo¹⁷⁻¹⁹. In Rene et al, the authors show that a minor substitution of a phenylsulfonamide for a benzylsulfonamide within the same chemical scaffold changes the compound from an inverse agonist to an agonist on RORγt with no activity on RORα¹⁹. Co-crystal structures of the benzylsulfonamide and phenylsulfonamide derivatives bound to RORγt provided further structural insights into the opposing MOA of these compounds. These studies clearly demonstrate that it is possible to upregulate basal RORγt activity with synthetic modulators.

### SUMMARY

Our recent efforts to optimize the SR1078 scaffold provided many analogs with improved biochemical and physiochemical properties. These compounds were evaluated for their ability to positively modulate IL17 to aid activation of T_{H}17 cells and for their ability to impact PD-1 cell surface expression. Here we show that activation of RORγt with the SR1078 analog SR0987, leads to increased expression of IL17 while repressing the expression of the checkpoint receptor PD-1, activities that the recently identified endogenous sterol agonists do not engender.

Accordingly, the invention provides, in various embodiments, a method for enhancing immunity in a patient, comprising administering to the patient an effective amount of an agonist of RORγt comprising a compound of formula (I)

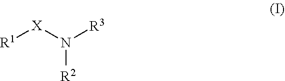

wherein X is C(O) or S(O)₂;
R¹ is phenyl, mono- or independently multi-substituted with J¹;
R² is H or alkyl, wherein any alkyl is optionally mono- or independently multi-substituted with J²;
R³ is phenyl wherein R substituted with J comprises

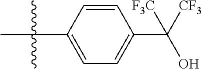 [SMILES: *c1ccc(C(O)(C(F)(F)F)C(F)(F)F)cc1]

or an alkyl, aryl, or arylalkyl ester of the hydroxyl group thereof, or an alkyl, aryl, or arylalkyl ether of the hydroxyl group thereof, wherein a wavy line indicates a point of attachment of J-substituted R³ to the nitrogen atom bearing R³.
J¹ when present is halo, cyano, nitro, alkoxy, or haloalkoxy; unsubstituted or substituted alkyl, haloalkyl, alkylcarboxamido, arylcarboxamido, or alkoxycarbonyl; unsubstituted or substituted aryl; unsubstituted or substituted arylsulfonyl; unsubstituted or substituted heteroaryl; unsubstituted or substituted heteroarylsulfonyl; or unsubstituted or substituted arylsulfonamido;
J² when present is halo, cyano, nitro, alkoxy, or haloalkoxy; unsubstituted or substituted alkyl, haloalkyl, alkylcarboxamido, arylcarboxamido or alkoxycarbonyl; unsubstituted or substituted aryl; unsubstituted or substituted arylsulfonyl; unsubstituted or substituted heteroaryl; unsubstituted or substituted heteroarylsulfonyl; or unsubstituted or substituted arylsulfonamido;
including any stereoisomer thereof, or any salt, solvate, hydrate, metabolite, or prodrug thereof.

For instance, administration of an effective amount of an agonist of RORγt comprising a compound of formula (I) can increase production of IL17 in situ, which can be associated with an increase in immunity in a patient.

The invention further provides a method of treating a cancer, comprising administering to a patient afflicted therewith an effective amount of an agonist of RORγt comprising a compound of formula (I).

For practice of the methods of the invention, the compound of formula (I) can be SR0987,

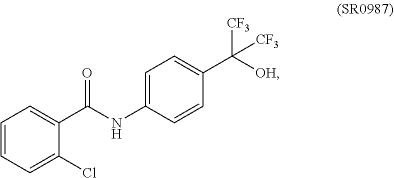 [SMILES: O=C(Nc1ccc(C(O)(C(F)(F)F)C(F)(F)F)cc1)c1ccccc1Cl]

as described herein.

For practice of the methods of the invention, the compound of formula (I) can be any of the compounds shown in Table 3, below.

### BRIEF DESCRIPTION OF THE FIGURES

FIG. 1. In vitro characterization of synthetic RORγ agonist and endogenous ligand, a) compound structure. b) RORγt agonist transactivation. Activation of Gal4-RORγ::UAS-Luc reporter assay for SR1078, SR0987, and desmosterol at a 30 μM concentration. c) CRC for SR0987 and desmosterol in the presence of ursolic acid (2 μM) in the Gal4-RORγ::UAS-Luc reporter assay in HEK293T cells (right panel), All error bars denote s.e.m. d) Activation of full-length RORγ in the presence of ursolic acid (2 μM) in HEK293T cells and co-transfected with SXRORE-Luc reporter. e) Activation of full-length RORγ receptor in the presence of ursolic acid (2 μM) in HEK293T cells and co-transfected with IL17-Luc reporter.

FIG. 2. Decreasing PD-1 by synthetic RORγ agonist. a) IL17A, PD-1 and granzyme B mRNA expression in stimulated EL4 cells (activated with PMA/lonomycin for 5 hr). b) PD-1 surface expression in EL4 cells. Cells were pretreated with compound (desmoterol, SR0987) for 48 hr, c) intracellular staining of IL17A in T_{H}17 cells, d) cell surface expression of PD-1. e) CD62L-PD1-cell population. f) PD-1 expression in human Jurkat T cells.

FIG. 3. a) Differential HDX kinetics of RORγt LBD±compounds plotted over the crystal structure PDB:3LOL. b) Differential HDX kinetics of ursolic acid treated RORγt LBD±compounds plotted over the crystal structure PDB:3LOL. Cool colors are increased protection to solvent exchange (increased stabilization) and warm colors are decreased protection to solvent exchange (decreased stabilization). Grey color represents no statistically significant change with and without ligands.

### DETAILED DESCRIPTION

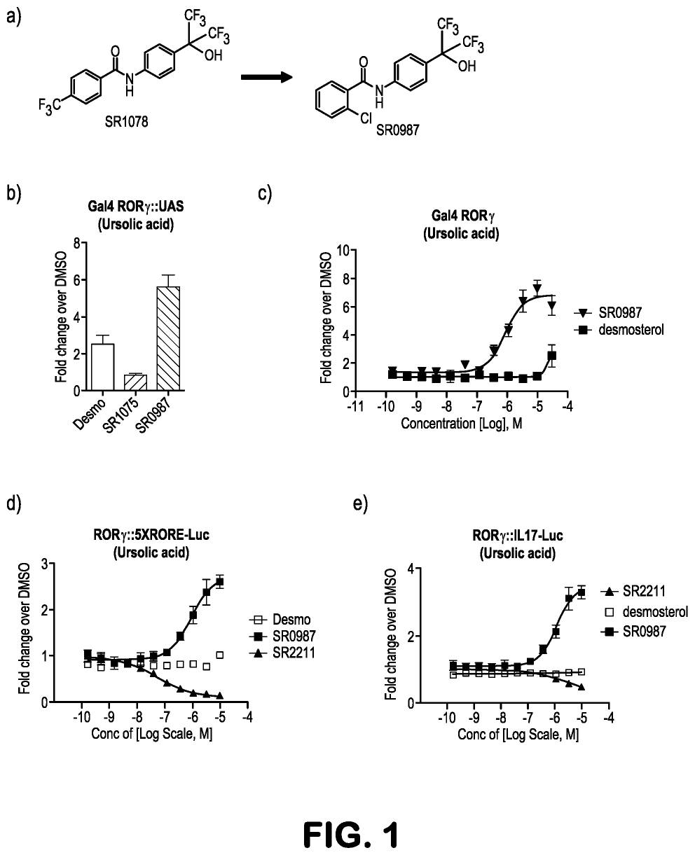

Competitive radioligand binding assays illustrated direct binding of SR1078 to the ligand binding domain (LBD) of RORγt albeit with weak affinity (IC₅₀˜15 μM)¹⁸. SR1078 was also shown to have direct interaction with RORγt via thermal shift assay as measured by Circular Dichroism (CD)¹⁷. Initial SAR of the benzamide ring suggests that substituents are tolerated at the ortho-position leading to SR0987 (FIG. 1a).

Compounds were subsequently screened in a Gal4 UAS-Luc cotransfection system in order to determine their ability to modulate RORγ activity in a cellular environment. Given that RORγt has high basal activity when expressed in cells, repression by the receptors' activity using an inverse agonist (e.g., ursolic acid) followed by test compound treatment offered the best window to detect agonism 3. Here cells were pre-treated with 2 μM ursolic acid (IC50˜0.8 μM) which afforded approximately 60-70% of RORγt activity prior to the addition of test compounds. Desmosterol was used as a control for agonism as it was recently identified as a putative endogenous agonist for RORγt capable of restoring RORγt activity in the presence of ursolic acid. Importantly, in this assay format, the potent inverse agonist SR2211^{20,21} demonstrated the ability to further repress the expression of the luciferase reporter gene in the presence of ursolic acid.

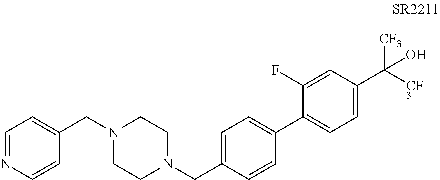 [SMILES: OC(c1ccc(-c2ccc(CN3CCN(Cc4ccncc4)CC3)cc2)c(F)c1)(C(F)(F)F)C(F)(F)F]

Initial screening of compounds was performed at a single concentration of 30 μM looking for compounds with improved reporter gene expression relative to desmosterol. In this screening format SR0987 afforded the highest fold induction of reporter gene expression (˜6 fold), whereas desmosterol and SR1078 resulted in only a minor induction of luciferase expression (≥2 fold) (FIG. 1b). Furthermore, SR0987 clearly shows a concentration dependent induction of reporter gene expression with an EC50 of ˜800 nM (FIG. 1c), Interestingly, desmosterol only induced luciferase expression at the highest concentration tested (˜2 fold at 30 μM). The concentration response curve for SR1078 confirms the improved agonist activity of SR0987. In addition, SR1078 and SR0987 demonstrate concentration dependent increase in interaction of RORγt with the SRC1-3 NR box peptide further validating that these compounds drive the agonist conformation of the receptor. As expected, SR2211 decreases interaction with this co-activator peptide in a concentration dependent fashion.

In order to determine if these compounds could modulate RORγt activity in the context of the full-length receptor, we used a co-transfection system in HEK293T cells in which full length RORγt was co-transfected along with a luciferase reporter under the control of either a basic promoter containing five copies of an ROR response element (5×RORE) or a minimal IL17 promoter. For all subsequent in vitro pharmacology studies we focused on the more efficacious synthetic agonist SR0987. As shown in FIGS. 1d and 1e, SR0987 demonstrated a concentration-dependent induction of reporter gene expression in both the 5×RORE and IL17 promoter transfected cells in the presence of full-length RORγt whereas minimal induction of the reporter gene was observed with desmosterol treatment. As expected, the inverse agonist SR2211 repressed both promoters in a concentration-dependent manner.

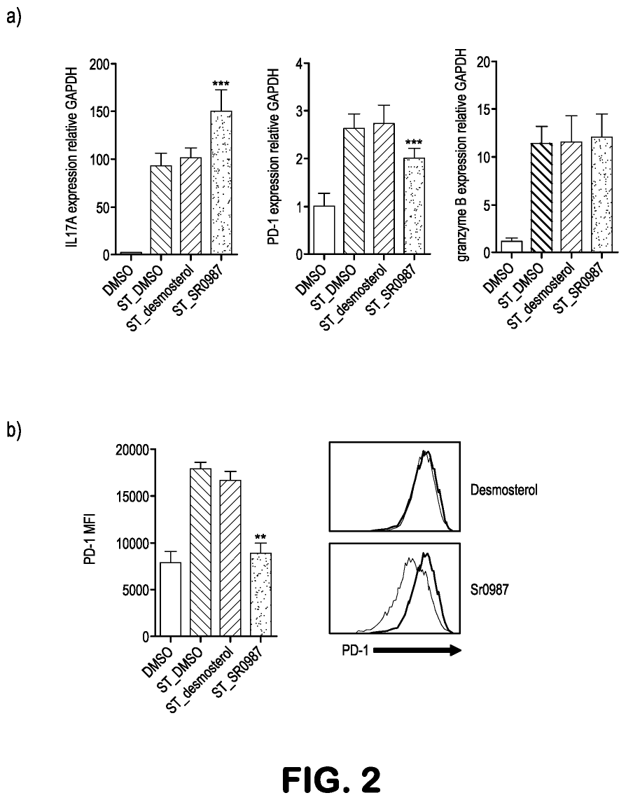

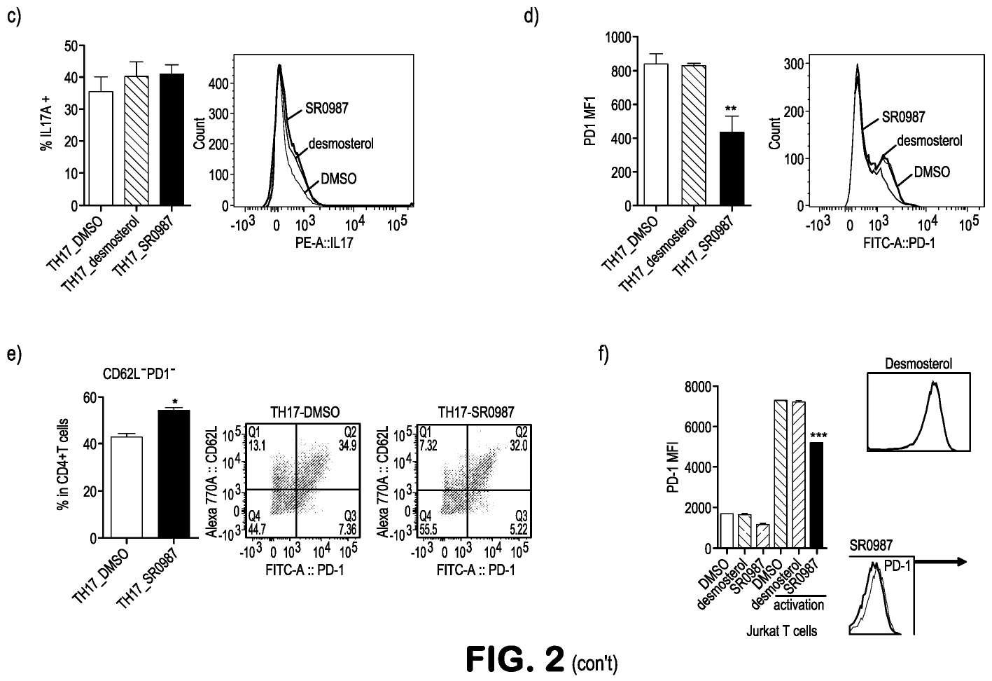

PD-1 is not expressed on resting T cells but its expression is induced within 24 hours after T cell receptor stimulation Z and is involved in the establishment and maintenance of immunological tolerance in the spontaneous development of autoimmune diseases by PD-1 deficient mice 14 Given that PD-L1 is expressed on various tumor cells and PD-1 expression is upregulated and sustained on T cells, it is clear that the PD-1/PD-L1 pathway plays an important role in tumor immunity. Here we used murine EL4 T lymphocytes or human Jurkat T cells as model systems to analyze gene expression upon T cell activation. Following treatment of cells with Phorbol 12-myristate 13-acetate (PMA) and ionomycin, the expression of granzyme B (cytotoxicity marker), PD-1 (immune checkpoint) and the RORγt target gene IL17 were analyzed by qPCR. As shown in FIG. 2a stimulation of EL4 cells led to an increase in the expression of all three genes and when coupled with treatment with the synthetic RORγt agonist SR0987 a further increase in expression of IL17 was observed suggesting that there was in induction of T cell activation. Surprisingly and unexpected, treatment with SR0987 led to a decrease in expression of PD-1. Compound treatment did not impact the expression of granzyme B. Combined these results suggest that treatment with SR0987 may enhance protective immunity by regulating expression of IL17 and PD-1 while maintaining the cytotoxic ability of these cells.

Using flow cytometery surface PD-1 expression was analyzed to determine if the decrease in gene expression of PD-1 correlates to a decrease of the protein on the cell surface, Cell: surface PD-1 expression was measured in murine and human T cell lines as well as in ex vivo differentiated murine T_{H}17 cells. SR0987 treatment resulted in a statistically significant reduction of the surface expression of PD-1 whereas desmostrol treatment showed no effect (FIG. 2b). Next we examined the impact of compound treatment on differentiated murine T_{H}17 cells. Treatment with SR0987 and or desmosterol resulted in a trend towards increased IL17 production (FIG. 2c). However, in this system SR0987 again demonstrated the ability to repress surface PD-1 expression whereas desmosterol had no effect (FIG. 2d). To determine if there was an increase in the active T cell population during T_{H}17 cell differentiation, the population of CD62L⁻PD1⁻ double negative cells was measured using flow cytometry. Naïve CD4+ T cells isolated from mice were differentiated using a cytokine cocktail in the presence or absence of ursolic acid. SR0987 resulted in a statistically significant increase in the CD62L⁻PD1⁻CD4⁺ cell population as compared when compared to DMSO treated cells (FIG. 2e). To determine if the effects of SR0987 on PD-1 expression would be observed in a human cell line, Jurkat T cells were treated with the compound. As shown in FIG. 2f, exposure of Jurkat T cells to SR0987 resulted in decreased cell surface PD-1 expression (FIG. 2f).

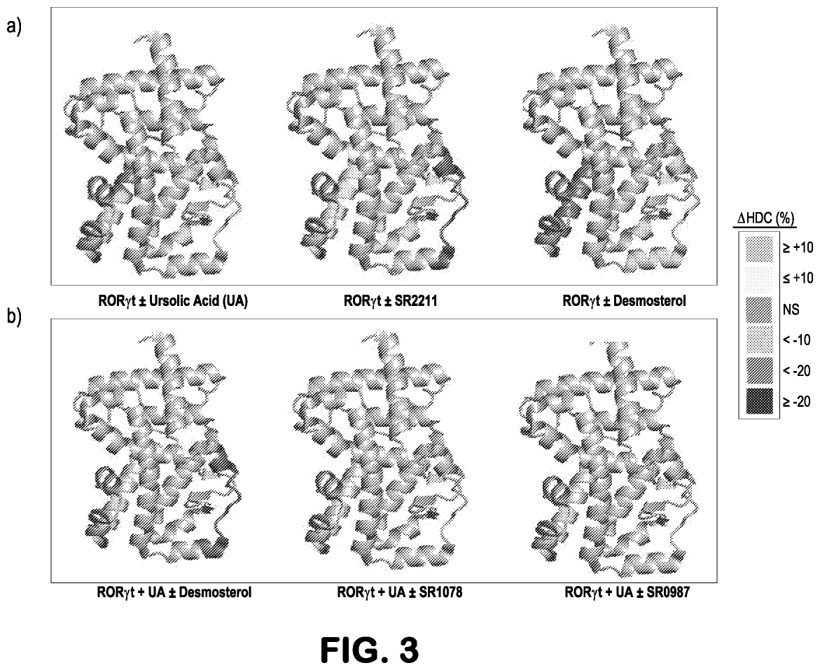

Taken together, these results suggest that SR0987 acts as a RORγt agonist and that use of such synthetic ligands may enhance immune response in the context of cancer. While the mechanism of action of RORγt agonists on regulation of the immune checkpoint receptor PD-1 is unclear, a correlation between RORγt and PD-1 expression has been observed in PD-1 knockout mice Z Regardless, to gain insights into the structural mechanism for agonist activity we examined the impact of putative agonist ligands on the conformational dynamics of RORγt. To achieve this, we utilized differential hydrogen/deuterium exchange (HDX) mass spectrometry. Previously, we have demonstrated the utility of HDX to monitor ligand-induced conformational changes in NRs including RORγt²⁶⁻²⁸. The differential HDX kinetics of RORγt LBD in the presence and absence of ursolic acid (inverse agonist), SR2211 (inverse agonist), and desmosterol (putative endogenous agonist) are shown overlaid on the 25-α-OHC:RORγ co-crystal structure (PDB ID: 3L0L)²⁸ (FIG. 3a) HDX revealed that helices 11 (H11) shows increased protection to solvent exchange (stabilization) with all RORγt ligands tested, suggesting common sites of direct interaction for ligands within the ligand-binding pocket (LBP) of RORγt. No statistically significant change in HDX kinetics was observed in the activation function-2 helix, helix 12 (H12), for these three complexes (Table 1a). In contrast, FIG. 3b shows differential HDX kinetics of RORγt exposed to ursolic acid followed by addition of desmosterol (putative endogenous agonist), SR1078 (agonist) and SR0987 (agonist) also overlaid on PDB ID: 3LOL. Protection to solvent exchange was again observed in H11; In addition, treatment with either SR1078 or SR0987, induced protection to solvent exchange in H12 that was not observed with desmosterol (Table 1b). This observation is consistent with the concentration-dependent activation of RORγt observed in cells with these two synthetic agonists. Similar agonist induced H12 protections have been previously observed with other NRs such as PPARγ²⁶. The differential patterns of H12 protection seen between agonists and inverse agonist are in line with the recently published crystal structures of RORγt-LBD in complex with synthetic inverse agonist and a synthetic agonist (PDB ID: 4WQP and 4WPF)¹⁹. These structures revealed that synthetic agonists pack against H3 and H11/12 interface and engages with RORγt LBD residues W317 (H3), H479 (H11) and Y502 (H12) resulting in a stable H12 conformation through a direct hydrogen bond between H479 and Y502 side chains, Whereas a synthetic inverse agonist dislodges H479 side chain into an orientation that is unfavorable for forming the hydrogen bond with Y502, which destabilized H12 (disordered in the structure) and disrupted co-activator interactions. Collectively, the HDX studies provide a structural basis for the agonist properties of SR0987.

Enhanced immunity and blockage of immune checkpoints has transformed cancer treatment with therapies targeting PD-1 showing unprecedented rates of durable clinical responses in patients with various cancers. The results presented here suggest that RORγ agonists may enhance T cell activation while repressing PD-1 without reducing the cytotoxic activity of these cells. Therefore, RORγt agonists can provide a unique combination therapy with approved anti-PD-1 molecules for treatment of cancer and can provide utility in the context of anti-PD-1 resistance.

TABLE 1a
Diffrential HDX Kinetics of RORγt ± Compounds (peptide sequences shown: SEQ ID NOs: 8-38)

| | | | | RORγt ± Ursolic | RORγt ± | RORγt ± | |
|---|---|---|---|---|---|---|---|
| Peptide Sequence | Charge | Start | | Acid (UA) | SR2211 | Desmosterol | Structure |
| TEIEHLVQ | 268 | 278 | 2 | −2(4)* | −3(4)* | −2(5)* | H1 |
| TEIEHLVQSVC | 268 | 278 | 2 | −3(4)* | −3(5)* | −1(4)* | H1 |
| TEIEHLVQSVCXS | 268 | 280 | 3 | 2(3)* | −2(3)* | 0(5)* | H1 |
| TEIEHLVQSVCKSYRETCQ | 268 | 286 | 2 | −5(3)* | −7(3)* | −5(3) | H1 |
| LRLEDL | 287 | 292 | 2 | 1(4)* | −7(4)* | −5(4)* | H2 |
| RLEDLL | 288 | 293 | 2 | 1(4)* | −8(3)* | −6(3) | H2 |
| RLEDLLRQRSNIFSRE | 288 | 303 | 4 | 0(3)* | −13(4)* | −10(4) | H2/H3 |
| EVTGYQRKS [symbol] | 304 | 316 | 2 | −1(3)* | −5(2)* | −7(3) | H3/H4 |
| WERCAHHLTEAIQ | 317 | 329 | 2 | −5(1)* | −6(1)* | −5(1)* | H4 |
| WERCAHHTEAIQY | 317 | 330 | 3 | −4(1)* | −4(1)* | −4(1)* | H4 |
| WEGAKRLSGF | 331 | 341 | 2 | −2(1)* | −1(2)* | −1(1)* | H4/H5 |
| FA [symbol] RLSGF | 334 | 341 | 2 | −3(1)* | −1(2)* | −1(2)* | H4/H5 |
| AKRLSGF | 335 | 341 | 2 | −3(2)* | −1(3)* | −1(2)* | H4/H5 |
| CQNDQVL | 345 | 352 | 1 | 0(1)* | 0(1)* | 0(1)* | H5/H6 |
| VRMCRAYNAENRTVF | 363 | 377 | 3 | −9(2) | −7(2)* | −7(2) | H6/B1 |
| CRAYCAONRTVF | 366 | 377 | 2 | −6(2)* | −5(1)* | −5(2) | B1 |
| FEGKYGGMEL | 373 | 387 | 2 | −9(2)* | −5(2)* | −6(3) | B2/H7 |
| FRALGCSE | 386 | 398 | 2 | −4(3)* | −6(2)* | −4(3)* | H7/H8 |
| LISSIFDFSHSLSAL | 395 | 410 | 2 | −5(2)* | −2(2)* | −4(2)* | H8 |
| ISSIFD | 397 | 402 | 1 | −3(1)* | 2(2)* | −4(2)* | H8 |
| ISSIFDFSHSL | 397 | 407 | 2 | −4(3)* | −2(3)* | −4(2)* | H8 |
| ISSIFDSHSLSAL | 397 | 410 | 2 | −6(2)* | −3(2)* | −4(2)* | H8 |
| DFSHSLSAL | 402 | 410 | 1 | −9(2)* | −4(2)* | −5(3) | H8 |
| FSHSLSAL | 403 | 410 | 2 | −7(2)* | −3(2)* | −4(3)* | H8 |
| HFSEDEIAL | 411 | 419 | 2 | 0(2)* | −1(3)* | −1(1)* | H9 |
| LAFHHHLCKTHRQSL | 448 | 463 | 4 | −3(1)* | −3(1)* | −3(2)* | H10 |
| AKLPFXGKLRSLCSQ | 464 | 478 | 3 | −25(4)* | −25(4)* | −12(4) | H11 |
| HVERLQFQHLHPIVVQ | 479 | 495 | 2 | −24(4)* | −25(4)* | −12(4) | H11 |
| QFQHLHPIVVQ | 484 | 495 | 2 | −12(3)* | −12(3)* | −6(4)* | H11 |
| AAFPPLYXEL | 495 | 505 | 2 | −0(3)* | 0(3)* | −2(4)* | H12 |
| AAFPPLYZELF | 496 | 506 | 2 | −0(3)* | 0(3)* | −3(3)* | H12 |

[symbol]

indicates data missing or illegible when filed

TABLE 1b
Diffrential HDX Kinetics of RORγt pretreated with ursolic acid (UA) ±
Compound (peptide sequences shown: SEQ ID NOs: 39-71)

| Peptide | | | | RORγt/UA ± | RORγt/UA ± | RORγt/UA ± | |
|---|---|---|---|---|---|---|---|
| Sequence | Start | End | Charge | Desmosterol | SR1078 | SR3-987 | Structure |
| TEIEHLVQ | 268 | 275 | 2 | −1(4)* | −2(3)* | −1(4)* | H1 |
| TEIEHLVQSVC | 268 | 278 | 2 | −2(4)* | 0(3)* | 0(4)* | H1 |
| TEIEHLVQSVCKS | 268 | 280 | 3 | 2(2)* | 1(4)* | 2(3)* | H1 |
| TEIEHLVQSVCKSYRETCQ | 268 | 286 | 3 | −1(3)* | 0(3)* | 0(2)* | H1 |
| TEIEHLVQSVCKSYRETCQ | 268 | 286 | 4 | 0(3)* | 1(3)* | 0(3)* | H1 |
| TEIEHLVQSVCKSYRETCQL | 286 | 287 | 3 | −3(4)* | 0(4)* | 0(3)* | H1 |
| RLEDL | 288 | 292 | 1 | 10(2)* | −2(4)* | −1(4)* | H2 |
| RLEDLL | 288 | 293 | 1 | 10(2)* | −3(5)* | −2(4)* | H2 |
| RLEDLLRQRS [symbol] FSRE | 289 | 303 | 4 | −13(3)* | −1(4)* | −1(4)* | H2/H3 |
| EVTGYQRKSMWEM | 304 | 316 | 2 | −6(3)* | 1(4)* | 0(3)* | H3/H4 |
| WERCAHHLTEAIQ | 317 | 329 | 3 | −1(1)* | 0(1)* | 0(1)* | H4 |
| WERCAHHTEAIQY | 317 | 330 | 3 | 0(1)* | 1(1)* | 0(1)* | H4 |
| WEGAKRLSGF | 331 | 341 | 2 | −1(2)* | 0(2)* | 0(1)* | H4/H5 |
| FAKRLSGF | 334 | 341 | 2 | 0(2)* | 0(2)* | 0(1)* | H4/H5 |
| CQNDQM | 345 | 352 | 1 | 0(1)* | 0(1)* | −1(2)* | H5/H6 |
| VRMCRAYNADNRTVF | 383 | 377 | 3 | −1(1)* | 1(1)* | 0(1)* | H6/B1 |
| CRAYNADNRTVF | 368 | 377 | 2 | 0(2)* | 2(1)* | 1(2)* | B1 |
| YNADNRTVF | 369 | 377 | 2 | −1(3)* | 1(3)* | 0(2)* | B1 |
| FEGKYGGMEL | 378 | 387 | 2 | −1(3)* | 2(3)* | 0(2)* | B2/H7 |
| FRALGCSEL | 388 | 395 | 2 | −1(2)* | −1(2)* | −2(2)* | H7/H8 |
| LISSIFDFSHSLSAL | 396 | 410 | 2 | 0(2)* | 2(2)* | 1(2)* | H8 |
| ISSIFD | 397 | 407 | 1 | −3(2)* | 1(2)* | 0(1)* | H8 |
| ISSIFDFSHSLSAL | 397 | 410 | 2 | 0(2)* | 2(2)* | 1(1)* | H8 |
| IFDFSHSLSAL | 400 | 410 | 2 | 4(3)* | 3(2)* | 2(1)* | H8 |
| DFSHSKSAL | 402 | 410 | 2 | 3(2)* | 2(2)* | 2(2)* | H8 |
| FSHSLSAL | 403 | 410 | 2 | 0(3)* | 2(3)* | 1(2)* | H8 |
| HFSEDEIAL | 411 | 410 | 2 | −1(2)* | 0(1)* | 0(1)* | H9 |
| LAFHHHL [symbol] THRQSIL | 448 | 463 | 4 | −2(1)* | 0(2)* | −1(1)* | H10 |
| AXLPPXGKLRSLCSQ | 454 | 478 | 3 | −1(4)* | 0(4)* | −2(3)* | H11 |
| HVERLQIFQHLHPIWQ | 479 | 495 | 2 | −6(3)* | −6(3)* | −6(2)* | H11 |
| QIFQHLHPIWQ | 464 | 495 | 2 | −3(3)* | −10(3)* | −11(2)* | H11 |
| AAFPPLYKEL | 496 | 505 | 2 | −4(2)* | −7(2)* | −7(3)* | H12 |
| AAFPPLYKELF | 496 | 506 | 2 | −3(2)* | −5(2)* | −6(3)* | H12 |

[symbol]

indicates data missing or illegible when filed

TABLE 2
Primer sequences for Q-PCR analysis

| Gene name | Primer sequence |
|---|---|
| Granzyme B | CCT CCT GCT ACT GCT GAC |
| (forward) | (SEQ ID NO: 1) |
| Granzyme B | GTC AGC ACA AAG TCC TCT |
| (reverse) | C (SEQ ID NO: 2) |
| IL17A (forward) | CTC CAG AAG GCC CTC AGA |
| | CTA C (SEQ ID NO: 3) |
| IL17A (reverse) | GGG TCT TCA TTG CGG TGG |
| | (SEQ ID NO: 4) |
| PD-1 (forward) | CGT CCC TCA GTC AAG AGG |
| | AG (SEQ ID NO: 5) |
| PD-1 (reverse) | GTC CCT AGA AGT GCC CAA |
| | CA (SEQ ID NO: 6) |

### EXAMPLES

TABLE 3
Compounds for practice of a method of the invention

| 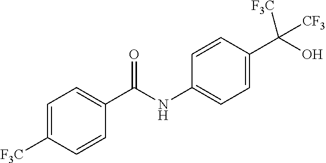 [SMILES: O=C(Nc1ccc(C(O)(C(F)(F)F)C(F)(F)F)cc1)c1ccc(C(F)(F)F)cc1] | 1: SR1078 |
|---|---|
| 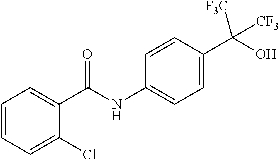 [SMILES: O=C(Nc1ccc(C(O)(C(F)(F)F)C(F)(F)F)cc1)c1ccccc1Cl] | 2: SR0987 |
| 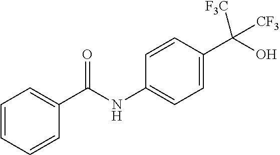 [SMILES: O=C(Nc1ccc(C(O)(C(F)(F)F)C(F)(F)F)cc1)c1ccccc1] | 3 |
| 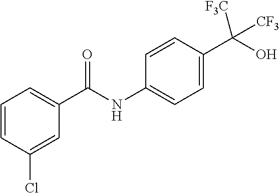 [SMILES: O=C(Nc1ccc(C(O)(C(F)(F)F)C(F)(F)F)cc1)c1cccc(Cl)c1] | 4 |
| 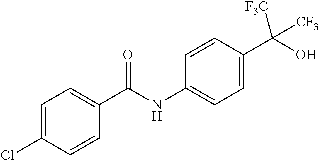 [SMILES: O=C(Nc1ccc(C(O)(C(F)(F)F)C(F)(F)F)cc1)c1ccc(Cl)cc1] | 5 |
| 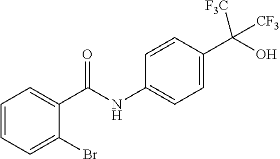 [SMILES: O=C(Nc1ccc(C(O)(C(F)(F)F)C(F)(F)F)cc1)c1ccccc1Br] | 6 |
| 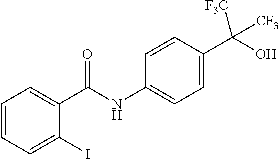 [SMILES: O=C(Nc1ccc(C(O)(C(F)(F)F)C(F)(F)F)cc1)c1ccccc1I] | 7 |
| 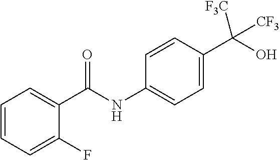 [SMILES: O=C(Nc1ccc(C(O)(C(F)(F)F)C(F)(F)F)cc1)c1ccccc1F] | 8 |
| 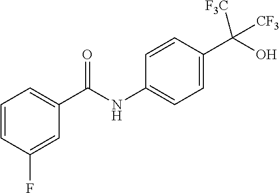 [SMILES: O=C(Nc1ccc(C(O)(C(F)(F)F)C(F)(F)F)cc1)c1cccc(F)c1] | 9 |
| 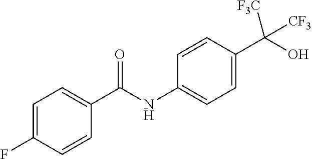 [SMILES: O=C(Nc1ccc(C(O)(C(F)(F)F)C(F)(F)F)cc1)c1ccc(F)cc1] | 10 |
| 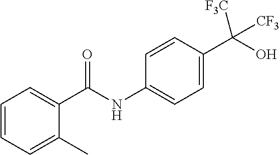 [SMILES: Cc1ccccc1C(=O)Nc1ccc(C(O)(C(F)(F)F)C(F)(F)F)cc1] | 11 |
| 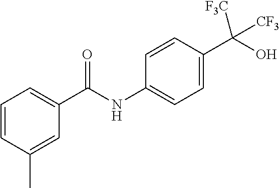 [SMILES: Cc1cccc(C(=O)Nc2ccc(C(O)(C(F)(F)F)C(F)(F)F)cc2)c1] | 12 |
| 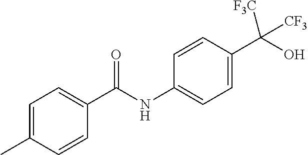 [SMILES: Cc1ccc(C(=O)Nc2ccc(C(O)(C(F)(F)F)C(F)(F)F)cc2)cc1] | 13 |
| 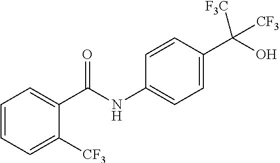 [SMILES: O=C(Nc1ccc(C(O)(C(F)(F)F)C(F)(F)F)cc1)c1ccccc1C(F)(F)F] | 14 |
| 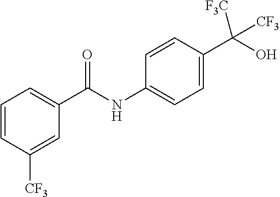 [SMILES: O=C(Nc1ccc(C(O)(C(F)(F)F)C(F)(F)F)cc1)c1cccc(C(F)(F)F)c1] | 15 |
| 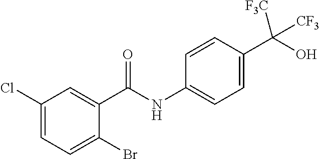 [SMILES: O=C(Nc1ccc(C(O)(C(F)(F)F)C(F)(F)F)cc1)c1cc(Cl)ccc1Br] | 16 |
| 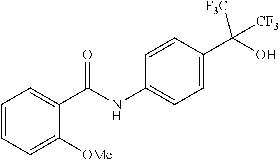 [SMILES: COc1ccccc1C(=O)Nc1ccc(C(O)(C(F)(F)F)C(F)(F)F)cc1] | 17 |
| 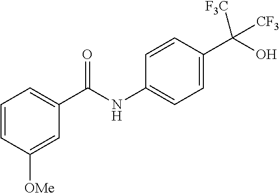 [SMILES: COc1cccc(C(=O)Nc2ccc(C(O)(C(F)(F)F)C(F)(F)F)cc2)c1] | 18 |
| 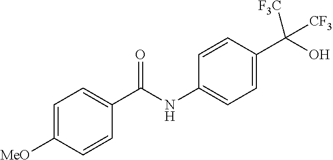 [SMILES: COc1ccc(C(=O)Nc2ccc(C(O)(C(F)(F)F)C(F)(F)F)cc2)cc1] | 19 |
| 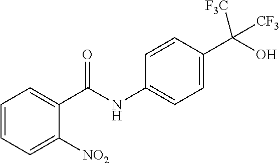 [SMILES: O=C(Nc1ccc(C(O)(C(F)(F)F)C(F)(F)F)cc1)c1ccccc1[N+](=O)[O-]] | 20 |
| 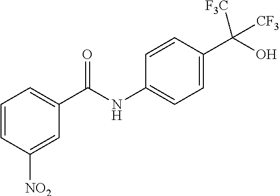 [SMILES: O=C(Nc1ccc(C(O)(C(F)(F)F)C(F)(F)F)cc1)c1cccc([N+](=O)[O-])c1] | 21 |
| 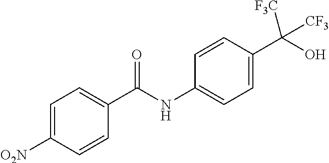 [SMILES: O=C(Nc1ccc(C(O)(C(F)(F)F)C(F)(F)F)cc1)c1ccc([N+](=O)[O-])cc1] | 22 |
| 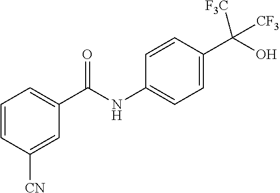 [SMILES: N#Cc1cccc(C(=O)Nc2ccc(C(O)(C(F)(F)F)C(F)(F)F)cc2)c1] | 23 |
| 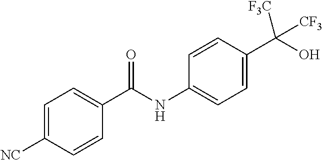 [SMILES: N#Cc1ccc(C(=O)Nc2ccc(C(O)(C(F)(F)F)C(F)(F)F)cc2)cc1] | 24 |
| 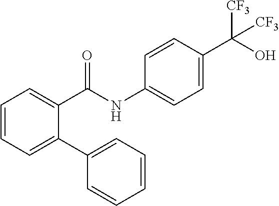 [SMILES: O=C(Nc1ccc(C(O)(C(F)(F)F)C(F)(F)F)cc1)c1ccccc1-c1ccccc1] | 25 |
| 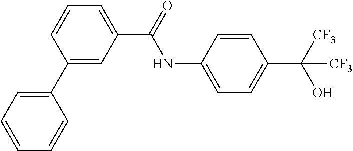 [SMILES: O=C(Nc1ccc(C(O)(C(F)(F)F)C(F)(F)F)cc1)c1cccc(-c2ccccc2)c1] | 26 |
| 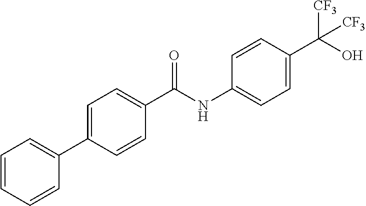 [SMILES: O=C(Nc1ccc(C(O)(C(F)(F)F)C(F)(F)F)cc1)c1ccc(-c2ccccc2)cc1] | 27 |
| 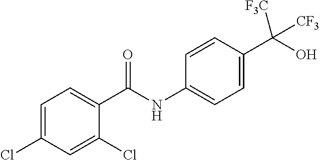 [SMILES: O=C(Nc1ccc(C(O)(C(F)(F)F)C(F)(F)F)cc1)c1ccc(Cl)cc1Cl] | 28 |
| 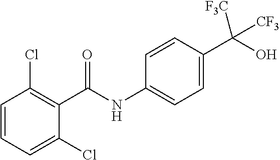 [SMILES: O=C(Nc1ccc(C(O)(C(F)(F)F)C(F)(F)F)cc1)c1c(Cl)cccc1Cl] | 29 |
| 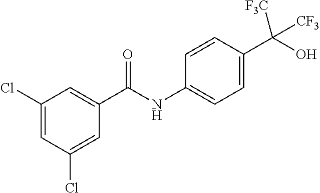 [SMILES: O=C(Nc1ccc(C(O)(C(F)(F)F)C(F)(F)F)cc1)c1cc(Cl)cc(Cl)c1] | 30 |
| 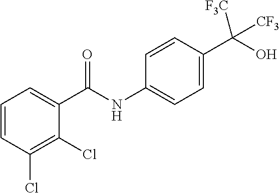 [SMILES: O=C(Nc1ccc(C(O)(C(F)(F)F)C(F)(F)F)cc1)c1cccc(Cl)c1Cl] | 31 |
| 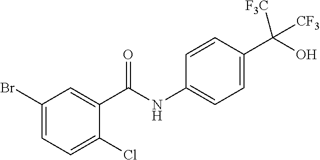 [SMILES: O=C(Nc1ccc(C(O)(C(F)(F)F)C(F)(F)F)cc1)c1cc(Br)ccc1Cl] | 32 |
| 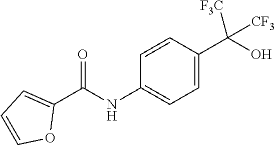 [SMILES: O=C(Nc1ccc(C(O)(C(F)(F)F)C(F)(F)F)cc1)c1ccco1] | 33 |
| 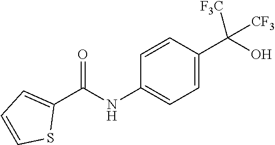 [SMILES: O=C(Nc1ccc(C(O)(C(F)(F)F)C(F)(F)F)cc1)c1cccs1] | 34 |
| 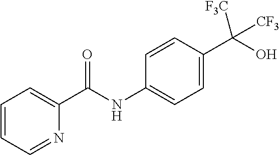 [SMILES: O=C(Nc1ccc(C(O)(C(F)(F)F)C(F)(F)F)cc1)c1ccccn1] | 35 |
| 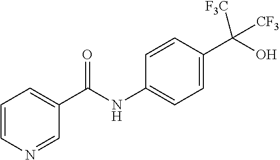 [SMILES: O=C(Nc1ccc(C(O)(C(F)(F)F)C(F)(F)F)cc1)c1cccnc1] | 36 |
| 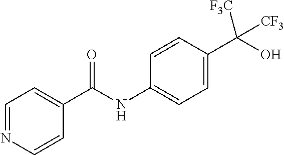 [SMILES: O=C(Nc1ccc(C(O)(C(F)(F)F)C(F)(F)F)cc1)c1ccncc1] | 37 |
| 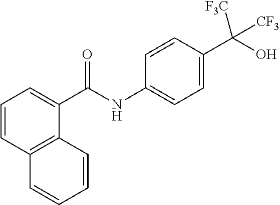 [SMILES: O=C(Nc1ccc(C(O)(C(F)(F)F)C(F)(F)F)cc1)c1cccc2ccccc12] | 38 |
| 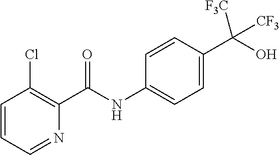 [SMILES: O=C(Nc1ccc(C(O)(C(F)(F)F)C(F)(F)F)cc1)c1ncccc1Cl] | 39 |
| 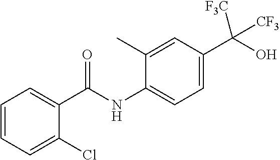 [SMILES: Cc1cc(C(O)(C(F)(F)F)C(F)(F)F)ccc1NC(=O)c1ccccc1Cl] | 40 |
| 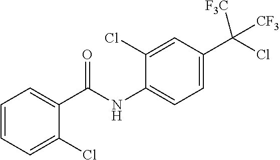 [SMILES: O=C(Nc1ccc(C(Cl)(C(F)(F)F)C(F)(F)F)cc1Cl)c1ccccc1Cl] | 41 |
| 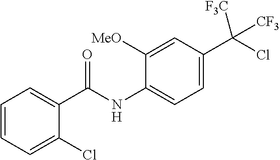 [SMILES: COc1cc(C(Cl)(C(F)(F)F)C(F)(F)F)ccc1NC(=O)c1ccccc1Cl] | 42 |
| 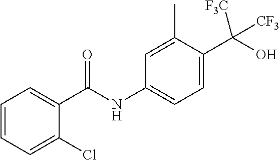 [SMILES: Cc1cc(NC(=O)c2ccccc2Cl)ccc1C(O)(C(F)(F)F)C(F)(F)F] | 43 |
| 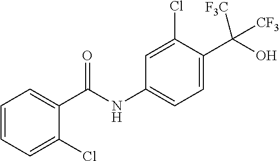 [SMILES: O=C(Nc1ccc(C(O)(C(F)(F)F)C(F)(F)F)c(Cl)c1)c1ccccc1Cl] | 44 |
| 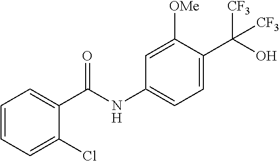 [SMILES: COc1cc(NC(=O)c2ccccc2Cl)ccc1C(O)(C(F)(F)F)C(F)(F)F] | 45 |
| 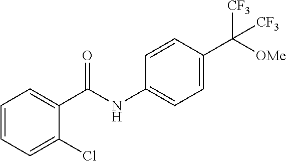 [SMILES: COC(c1ccc(NC(=O)c2ccccc2Cl)cc1)(C(F)(F)F)C(F)(F)F] | 46 |
| 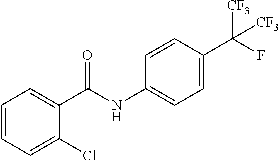 [SMILES: O=C(Nc1ccc(C(F)(C(F)(F)F)C(F)(F)F)cc1)c1ccccc1Cl] | 47 |
| 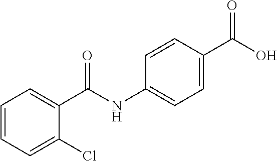 [SMILES: O=C(O)c1ccc(NC(=O)c2ccccc2Cl)cc1] | 48 |
| 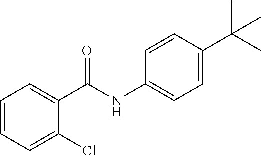 [SMILES: CC(C)(C)c1ccc(NC(=O)c2ccccc2Cl)cc1] | 49 |
| 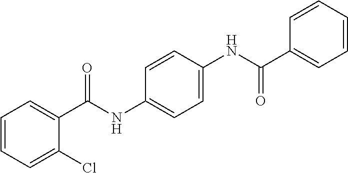 [SMILES: O=C(Nc1ccc(NC(=O)c2ccccc2Cl)cc1)c1ccccc1] | 50 |
| 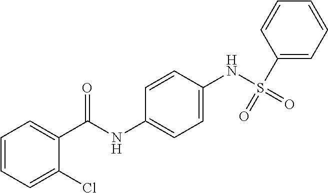 [SMILES: O=C(Nc1ccc(NS(=O)(=O)c2ccccc2)cc1)c1ccccc1Cl] | 51 |
| 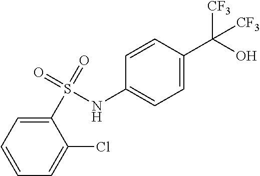 [SMILES: O=S(=O)(Nc1ccc(C(O)(C(F)(F)F)C(F)(F)F)cc1)c1ccccc1Cl] | 52 |
| 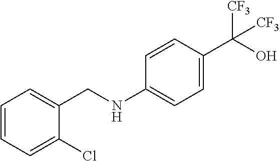 [SMILES: OC(c1ccc(NCc2ccccc2Cl)cc1)(C(F)(F)F)C(F)(F)F] | 53 |
| 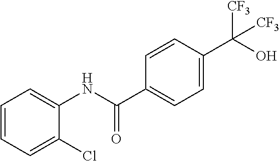 [SMILES: O=C(Nc1ccccc1Cl)c1ccc(C(O)(C(F)(F)F)C(F)(F)F)cc1] | 54 |
| 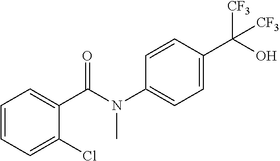 [SMILES: CN(C(=O)c1ccccc1Cl)c1ccc(C(O)(C(F)(F)F)C(F)(F)F)cc1] | 55 |

Compounds. Chemicals and solvents were purchased from commercial suppliers. Sterols were purchased from Avanti Polar Lipids and all other chemicals were purchased from Sigma. Compounds were purified using CombiFlash Rf 200 flash chromatography on silica gel on RediSEp Rf from Teledyme Isco, Inc. Yields refer to isolated compounds, estimated to be >98% pure as determined by 1H NMR or HPLC. Melting points were measured on a Stuart automatic melting point SMP40. ¹H, ¹³CNMR spectra were recorded on Bruker Spectrometer operating at 400 MHz and 101 MHz respectively. All chemical shift values, 6, and coupling constants, J, are quoted in ppm and Hz, respectively. Infra-Red spectrums were recorded on Perkin Elmer FT-IR Spectrometer. Synthesis of SR1078 was performed as previously described¹⁸.

### Synthetic Example 2 (SR987): 2-chloro-N-(4-(1,1,1,3,3,3-hexafluoro-hydroxypropan-2-yl)phenyl)benzamide

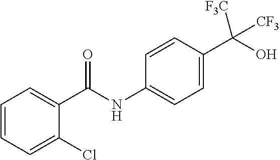 [SMILES: O=C(Nc1ccc(C(O)(C(F)(F)F)C(F)(F)F)cc1)c1ccccc1Cl]

To a solution of 4-(1-hydroxy-1-trifluoromethyl-2,2,2-trifluoroethyl)aniline (60 mg, 0.232 mmol) in CH₂Cl₂ (2 mL) were successively added at RT N,N-diisopropylethylamine (80 μL, 0.463 mmol) and 2-chlorobenzoyl chloride (41 μL, 0.324 mmol). The mixture was stirred for 3 h and concentrated under reduce pressure. The crude residue was directly purified by column chromatography on silica gel without any workup by hexane/AcOEt (8/2) to obtain 85 mg (71%) of SR987 as a white powder FTIR cm⁻¹ 3338, 3028, 1643, 1521, 1410, 1254, 1219, 1188, 1112, 968, 944, 826; ¹H NMR (400 MHz, MeOD-d4) δ=7.97 (t, J=1.8 Hz, 1H), 7.91-7.86 (m, 1H), 7.85-7.80 (m, 2H), 7.75-7.70 (m, 2H), 7.63-7.58 (m, 1H), 7.51 (t, J=7.8 Hz, 1H); ¹³C NMR (101 MHz, MeOD-d4) δ=167.5, 141.5, 138.2, 135.9, 133.1, 131.4, 129.0 (2C), 128.9, 128.5, 127.3 (2C), 121.7 There are tree carbons missing for the description of SR1078. They correspond to the three carbons of the (1-hydroxy-1-trifluoromethyl-2,2,2-trifluoroethyl) moiety. The fluorine coupling with these carbons give multiplets that are very difficult to see on the ¹³C spectrum even with a prolonged number of scans, HRMS (ESI) m/z [M+H⁺] calcd for C₁₆H₁₀ClF₆NO₂, 398.0377; found, 398.0395; Mp=170-172° C.

### Synthetic Example 3: N-(4-(1,1,1,3,3,3-hexafluoro-2-hydroxypropan-2-yl)phenyl)benzamide

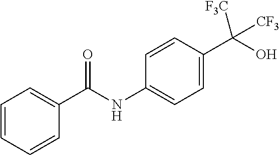 [SMILES: O=C(Nc1ccc(C(O)(C(F)(F)F)C(F)(F)F)cc1)c1ccccc1]

The title compound was prepared according to the Synthetic Example 2 from 4-(1-hydroxy-1-trifluoromethyl-2,2,2-trifluoroethyl)aniline (150 mg, 0.58 mmol) and benzoyl chloride (94 μL, 0.81 mmol). The crude product was purified by preparative HPLC (20-100% CH₃CN/MeOH (1:1) in H₂O (0.01% TFA)) which provided after lyophilization 174 mg (83%) of the title compound as a colorless solid; ¹H NMR (400 MHz, CDCl₃) δ=7.91 (br.s, 1H), 7.86-7.90 (m, 2H), 7.72-7.79 (m, 4H), 7.56-7.63 (m, 1H), 7.50-7.56 (m, 2H).

### Synthetic Example 4: 3-chloro-N-(4-(1,1,1,3,3,3-hexafluoro-2-hydroxypropan-2-yl)phenyl)benzamide

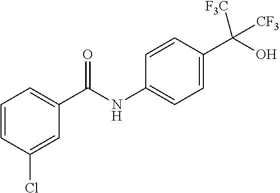 [SMILES: O=C(Nc1ccc(C(O)(C(F)(F)F)C(F)(F)F)cc1)c1cccc(Cl)c1]

The title compound was prepared according to the Synthetic Example 2 from 4-(1-hydroxy-1-trifluoromethyl-2,2,2-trifluoroethyl)aniline (50 mg, 0.19 mmol) and 3-chlorobenzoyl chloride (30 μL, 0.23 mmol). The crude product was purified by column chromatography on silica gel without any workup by hexane/AcOEt (8/2) to obtain 53 mg (69%) of the title compound as a white powder, ¹H NMR (400 MHz, MeOD-d4) 5=7.96 (t, J=1.64 Hz, 1H), 7.84-7.89 (m, 1H), 7.80-7.84 (m, 2H), 7.69-7.75 (m, 2H). 7.57 (m, 1H), 7.48 (t, J=7.89 Hz, 1H).

### Synthetic Example 5: 4-chloro-N-(4-(1,1,1,3,3,3-hexafluoro-2-hydroxypropan-2-yl)phenyl)benzamide

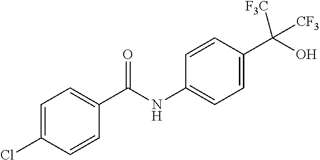 [SMILES: O=C(Nc1ccc(C(O)(C(F)(F)F)C(F)(F)F)cc1)c1ccc(Cl)cc1]

The title compound was prepared according to the Synthetic Example 2 from 4-(1-hydroxy-1-trifluoromethyl-2,2,2-trifluoroethyl)aniline (40 mg, 0.15 mmol) and 4-chlorobenzoyl chloride (24 μL, 0.18 mmol). The crude product was purified by column chromatography on silica gel without any workup by hexane/AcOEt (713) to obtain 46 mg (75%) of the title compound as a white powder. ¹H NMR (400 MHz, MeOD-d4) δ 7.90-7.95 (m, 2H), 7.78-7.85 (m, 2H), 7.69-7.75 (m, 2H), 7.49-7.56 (m, 2H).

### Synthetic Example 6: 2-bromo-N-(4-(1,1,1,3,3,3-hexafluoro-2-hydroxypropan-2-yl)phenyl)benzamide

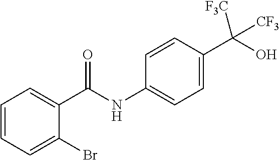 [SMILES: O=C(Nc1ccc(C(O)(C(F)(F)F)C(F)(F)F)cc1)c1ccccc1Br]

The title compound was prepared according to the Synthetic Example 2 from 4-(1-hydroxy-1-trifluoromethyl-2,2,2-trifluoroethyl)aniline (600 mg, 2.31 mmol) and 2-bromobenzoyl chloride (363 μL, 2.78 mmol). The crude product was purified by column chromatography on silica gel without any workup by hexane/AcOEt (8/2) to obtain 941 mg (92%) of the title compound as a white powder, ¹H NMR (400 MHz, CDCl₃) δ=7.76-7.82 (m, 2H), 7.67-775 (n, 3H), 7.55 (dd, J=1, 77, 7.58 Hz, 1H), 7.48 (dt, J=1.77, 7.58 Hz, 1H), 7.40 (dt, J=1.77, 7.58 Hz, 1H).

### Synthetic Example 7: 2-iodo-N-(4-(1,1,1,3,3,3-hexafluoro-2-hydroxypropan-2-yl)phenyl)benzamide

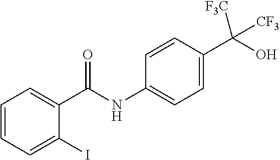 [SMILES: O=C(Nc1ccc(C(O)(C(F)(F)F)C(F)(F)F)cc1)c1ccccc1I]

The title compound was prepared according to the Synthetic Example 2 from 4-(1-hydroxy-1-trifluoromethyl-2,2,2-trifluoroethyl)aniline (300 mg, 1.16 mmol) and 2-iodobenzoyl chloride (345 mg, 1.39 mmol). The crude product was purified by column chromatography on silica gel without any workup by hexane/AcOEt (8/2) to obtain 539 mg (95%) of the title compound as a white powder. ¹H NMR (400 MHz, CDCl₃) δ=7.93 (dd, J=0.88, 7.96 Hz, 1H), 7.74-7.78 (m, 4H), 7.59 (br. s., 1H), 7.50-7.56 (m, 1H), 7.43-7.48 (m, 1H), 7.15-7.21 (m, 1H).

### Synthetic Example 8: 2-fluoro-N-(4-(1,1,1,1,3,3,3-hexafluoro-2-hydroxypropan-2-yl)phenyl)benzamide

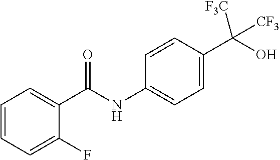 [SMILES: O=C(Nc1ccc(C(O)(C(F)(F)F)C(F)(F)F)cc1)c1ccccc1F]

The title compound was prepared according to the Synthetic Example 2 from 4-(1˜hydroxy-1-trifluoromethyl-2,2,2-trifluoroethyl)aniline (60 mg, 0.23 mmol) and 2-fluorobenzoyl chloride (39 μL, 0.32 mmol). The crude product was purified by preparative HPLC (20-100% CH₃CN/MeOH (1:1) in H₂O (0.01% TFA)) which provided after lyophilization 57 mg (65%) of the title compound as a colorless solid; ¹H NMR (400 MHz, CDCl₃) δ=8.56 (d, J=15.41 Hz, 1H), 8.19 (t, J=7.45 Hz, 1H), 7.69-7.82 (m, 4H), 7.51-7.61 (m, 1H), 7.35 (t, J=7.58 Hz, 1H), 7.21 (dd, J=7.58, 15.4 Hz, 1H).

### Synthetic Example 9: 3-fluoro-N-(4-(1,1,1,3,3,3-hexafluoro-2-hydroxypropan-2-yl)phenyl)benzamide

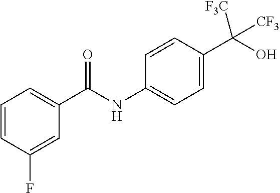 [SMILES: O=C(Nc1ccc(C(O)(C(F)(F)F)C(F)(F)F)cc1)c1cccc(F)c1]

The title compound was prepared according to the Synthetic Example 2 from 4-(1-hydroxy-1-trifluoromethyl-2,2,2-trifluoroethyl)aniline (60 mg, 0.23 mmol) and 3-fluorobenzoyl chloride (39 μL, 0.32 mmol). The crude product was purified by preparative HPLC (20-100% CH₃CN/MeOH (1:1) in H₂O (0.01% TFA)) which provided after lyophilization 66 mg (75%) of the title compound as a colorless solid; ¹H NMR (400 MHz, CDCl₃) δ=7.90 (br. s., 1H), 7.74-7.78 (m, 4H), 7.65-7.68 (m, 1H), 7.58-7.63 (m, 1H), 7.48-7.54 (m, 1H), 7.26-7.32 (m, 1H).

### Synthetic Example 10: 4-fluoro-N-(4-(1,1,1,3,3,3-hexafluoro-2-hydroxypropan-2-yl)phenyl)benzamide

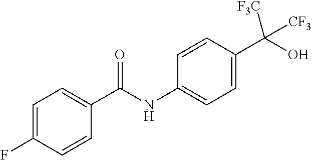 [SMILES: O=C(Nc1ccc(C(O)(C(F)(F)F)C(F)(F)F)cc1)c1ccc(F)cc1]

The title compound was prepared according to the Synthetic Example 2 from 4-(1-hydroxy-1-trifluoromethyl-2,2,2-trifluoroethyl)aniline (60 mg, 0.23 mmol) and 4-fluorobenzoyl chloride (38 μL, 0.32 mmol). The crude product was purified by preparative HPLC (20-100% CH₃CN/MeOH (1:1) in H₂O (0.01% TFA)) which provided after lyophilization 65 mg (74%) of the title compound as a colorless solid; ¹H NMR (400 MHz, CDC) δ=7.95 (br. s, 1H), 7.86-7.92 (m, 2H), 7.69-7.76 (m, 4H), 7.15-722 (m, 2H).

### Synthetic Example 11: N-(4-(1,1,1,3,3,3-hexafluoro-2-hydroxypropan-2-yl)phenyl)-2-methylbenzamide

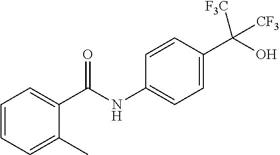 [SMILES: Cc1ccccc1C(=O)Nc1ccc(C(O)(C(F)(F)F)C(F)(F)F)cc1]

The title compound was prepared according to the Synthetic Example 2 from 4-(1-hydroxy-1-trifluoromethyl-2,2,2-trifluoroethyl)aniline (50 mg, 0.19 mmol) and o-toluoyl chloride (36 mg, 0.23 mmol). The crude product was purified by column chromatography on silica gel without any workup by hexane/AcOEt (812) to obtain 50 mg (69%) of the title compound as a white powder. ¹H NMR (400 MHz, MeOD-d4) δ=7.80 (d, J=8.99 Hz, 2H), 7.72 (d, J=8.77 Hz, 2H), 7.45-7.51 (m, 1H), 7.37 (m, 1H), 7.26-7.33 (m, 2H), 2.46 (s, 3H).

### Synthetic Example 12: N-(4-(1,1,1,3,3,3-hexafluoro-2-hydroxypropan-2-yl)phenyl)-3-methylbenzamide

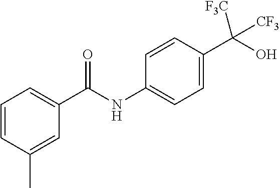 [SMILES: Cc1cccc(C(=O)Nc2ccc(C(O)(C(F)(F)F)C(F)(F)F)cc2)c1]

The title compound was prepared according to the Synthetic Example 2 from 4-(1-hydroxy-1-trifluoromethyl-2,2,2-trifluoroethyl)aniline (50 mg, 0.19 mmol) and m-toluoyl chloride (36 mg, 0.23 mmol). The crude product was purified by column chromatography on silica gel without any workup by hexane/AcOEt (8/2) to obtain 49 mg (67%) of the title compound as a white powder. 1H NMR (400 MHz, MeOD-d4) δ 7.80-7.85 (m, 2H), 7.77 (s, 1H), 7.69-7.75 (m, 3H), 7.36-7.43 (m, 2H), 2.43 (s, 3H).

### Synthetic Example 13: N-(4-(1,1,1,3,3,3-hexafluoro-2-hydroxypropan-2-yl)phenyl)-4-methylbenzamide

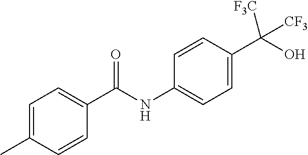 [SMILES: Cc1ccc(C(=O)Nc2ccc(C(O)(C(F)(F)F)C(F)(F)F)cc2)cc1]

The title compound was prepared according to the Synthetic Example 2 from 4-(1-hydroxy-1-trifluoromethyl-2,2,2-trifluoroethyl)aniline (50 mg, 0.19 mmol) and p-toluoyl chloride (31 μL, 0.23 mmol). The crude product was purified by preparative HPLC (20-100% CH₃CN/MeOH (1:1) in H₂O (0.01% TFA)) which provided after lyophilization 43 mg (63%) of the title compound as a colorless solid; ¹H NMR (400 MHz, MeOD-d4) δ=7.78-7.87 (m, 4H), 7.67-7.74 (m, 2H), 7.29-7.36 (m, 2H), 2.41 (s, 3H).

### Synthetic Example 14: N-(4-(1,1,1,3,3,3-hexafluoro-2-hydroxypropan-2-yl)phenyl)-2-(trifluoromethyl)benzamide

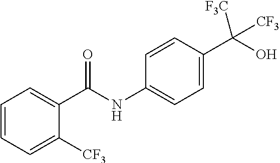 [SMILES: O=C(Nc1ccc(C(O)(C(F)(F)F)C(F)(F)F)cc1)c1ccccc1C(F)(F)F]

The title compound was prepared according to the Synthetic Example 2 from 4-(1-hydroxy-1-trifluoromethyl-2,2,2-trifluoroethyl)aniline (50 mg, 0.19 mmcl) and 2-trifluoromethylbenzoyl chloride (30 μL, 0.23 mmol). The crude product was purified by preparative HPLC (20-100% CH₃CN/MeOH (1:1) H₂O (0.01% TFA)) which provided after lyophilization 66 mg (79%) of the title compound as a colorless solid; ¹H NMR (400 MHz, MeOD-d4) δ=7.80-7.84 (m, 1H), 7.69-7.78 (m, 5H), 7.65-7.69 (m, 2H).

### Synthetic Example 15: N-(4-(1,1,1,3,3,3-hexafluoro-2-hydroxypropan-2-yl)phenyl)-3-(trifluoromethyl)benzamide

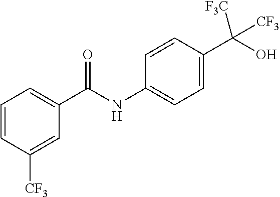 [SMILES: O=C(Nc1ccc(C(O)(C(F)(F)F)C(F)(F)F)cc1)c1cccc(C(F)(F)F)c1]

The title compound was prepared according to the Synthetic Example 2 from 4-(1-hydroxy-1-trifluoromethyl-2,2,2-trifluoroethyl)aniline (40 mg, 0.15 mmol) and 3-trifluoromethylbenzoyl chloride (25 μL, 0.18 mmol). The crude product was purified by column chromatography on silica gel without any workup by hexane/AcOEt (8/2) to obtain 47 mg (71%) of the title compound as a white powder. H NMR (400 MHz, MeOD-d4) δ=8.28 (s, 11H), 8.19-8.23 (m, 1H), 7.87-7.91 (m, 11H), 7.82-7.87 (m, 2H), 7.69-7.76 (m, 3H).

### Synthetic Example 16: 2-bromo-5-chloro-N-(4-(1,1,1,3,3,3-hexafluoro-2-hydroxypropan-2-yl)phenyl)benzamide

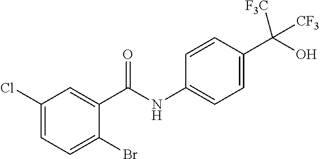 [SMILES: O=C(Nc1ccc(C(O)(C(F)(F)F)C(F)(F)F)cc1)c1cc(Cl)ccc1Br]

The title compound was prepared according to the Synthetic Example 2 from 4-(1-hydroxy-1-trifluoromethyl-2,2,2-trifluoroethyl)aniline (100 mg, 0.39 mmol) and 2-bromo-5-chlorobenzoyl chloride (120 mg, 046 mmol). The crude product was purified by column chromatography on silica gel without any workup by hexane/AcOEt (7/3) to obtain 130 mg (71%) of the title compound as a white powder: 1H NMR (400 MHz, CDCl₃) δ=8.12 (br. s., 1H), 7.67-7.76 (m, 4H), 7.51-7.58 (m, 2H), 7.26-7.31 (m, 1H).

### Synthetic Example 17: N-(4-(1,1,1,3,3,3-hexafluoro-2-hydroxypropan-2-yl)phenyl)-2-methoxybenzamide

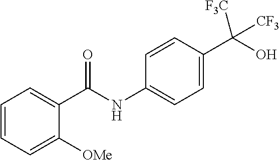 [SMILES: COc1ccccc1C(=O)Nc1ccc(C(O)(C(F)(F)F)C(F)(F)F)cc1]

The title compound was prepared according to the Synthetic Example 2 from 4-(1-hydroxy-1-trifluoromethyl-2,2,2-trifluoroethyl)aniline (40 mg, 0_15 mmol) and o-anisole chloride (28 μL, 0.18 mmol). The crude product was purified by column chromatography on silica gel without any workup by hexane/AcOEt (8/2) to obtain 60 mg (99%) of the title compound as a white powder ¹H NMR (400 MHz, MeOD-d4) δ=7.90 (d, J=7.89 Hz, 1H), 7.78-7.80 (m, 2H), 7.70-7.72 (m, 2H), 7.47-7.55 (m, 1H), 7.15 (d, J=8.11 Hz, 1H), 7.04-7.11 (m, 1H), 4.00 (s, 3H).

### Synthetic Example 18: N-(4-(1,1,1,3,3,3-hexafluoro-2-hydroxypropan-2-yl)phenyl)-3-methoxybenzamide

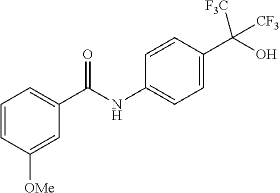 [SMILES: COc1cccc(C(=O)Nc2ccc(C(O)(C(F)(F)F)C(F)(F)F)cc2)c1]

The title compound was prepared according to the Synthetic Example 2 from 4-(1-hydroxy-1-trifluoromethyl-2,2,2-trifluoroethyl)aniline (40 mg, 0.15 mmol) and 3-methoxybenzoyl chloride (26 μL, 0.18 mmol). The crude product was purified by column chromatography on silica gel without any workup by hexane/AcOEt (8/2) to obtain 59 mg (97%) of the title compound as a white powder. ¹H NMR (400 MHz, MeOD-d4) δ=7.80-7.85 (m, 2H), 7.69-7.75 (m, 2H), 7.47-7.53 (m, 2H), 7.38-7.43 (m, 1H), 7.12-7.14 (m, 1H), 3.85 (s, 3H).

### Synthetic Example 19: N-(4-(1,1,1,3,3,3-hexafluoro-2-hydroxypropan-2-yl)phenyl)-4-methoxybenzamide

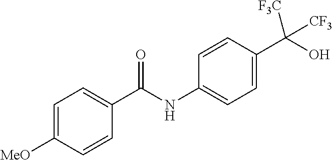 [SMILES: COc1ccc(C(=O)Nc2ccc(C(O)(C(F)(F)F)C(F)(F)F)cc2)cc1]

The title compound was prepared according to the Synthetic Example 2 from 4-(1-hydroxy-1-trifluoromethyl-2,2,2-trifluoroethyl)aniline (60 mg, 0.23 mmol) and 4-methoxybenzoyl chloride (44 μL, 0.32 mmol). The crude product was purified by preparative HPLC (20-100% CH₃CN/MeOH (1:1) in H₂O (0.01% TFA)) which provided after lyophilization 63 mg (69%) of the title compound as a colorless solid; ¹H NMR (400 MHz, CDCl₃) δ=7.83-7.90 (m, 2H), 7.82 (br. s, 1H), 7.70-7.78 (m, 4H), 6.98-7.04 (m, 2H), 3.90 (s, 3H).

### Synthetic Example 20: N-(4-(1,1,1,3,3,3-hexafluoro-2-hydroxypropan-2-yl)phenyl)-2-nitrobenzamide

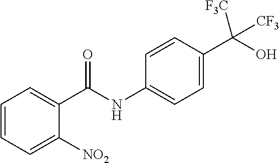 [SMILES: O=C(Nc1ccc(C(O)(C(F)(F)F)C(F)(F)F)cc1)c1ccccc1[N+](=O)[O-]]

The title compound was prepared according to the Synthetic Example 2 from 4-(1-hydroxy-1-trifluoromethyl-2,2,2-trifluoroethyl)aniline (40 mg, 0.15 mmol) and 2-nitrobenzoyl chloride (24 μL, 0.18 mmol). The crude product was purified by column chromatography on silica gel without any workup by hexane/AcOEt (8/2) to obtain 62 mg (98%) of the title compound as a white powder, ¹H NMR (400 MHz, MeOD-d4)=8.18 (dd, J=1.21, 8.44 Hz, 1H), 7.79-7.86 (m, 1H), 7.68-7.78 (m, 6H).

### Synthetic Example 21: N-(4-(1,1,1,3,3,3-hexafluoro-2-hydroxypropan-2-yl)phenyl)-3-nitrobenzamide

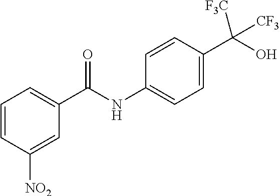 [SMILES: O=C(Nc1ccc(C(O)(C(F)(F)F)C(F)(F)F)cc1)c1cccc([N+](=O)[O-])c1]

The title compound was prepared according to the Synthetic Example 2 from 4-(1-hydroxy-1-trifluoromethyl-2,2,2-trifluoroethyl)aniline (40 mg, 0.15 mmol) and 3-nitrobenzoyl chloride (34 mg, 0.18 mmol). The crude product was purified by column chromatography on silica gel without any workup by hexane/AcOEt (8/2) to obtain 35 mg (56%) of the title compound as a white powder. ¹H NMR (400 MHz, MeOD-d4)=8.82 (t, J=1.86 Hz, 1H), 8.43-8.46 (m, 1H), 8.34-8.36 (m, 1H), 7.82-7.89 (m, 2H), 7.77-7.81 (m, 1H), 7.71-7.77 (m, 2H).

### Synthetic Example 22: N-(4-(1,1,1,3,3,3-hexafluoro-2-hydroxypropan-2-yl)phenyl)-4-nitrobenzamide

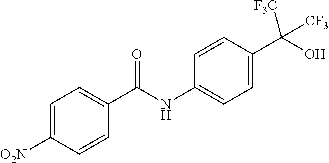 [SMILES: O=C(Nc1ccc(C(O)(C(F)(F)F)C(F)(F)F)cc1)c1ccc([N+](=O)[O-])cc1]

The title compound was prepared according to the Synthetic Example 2 from 4-(1-hydroxy-1-trifluoromethyl-2,2,2-trifluoroethyl)aniline (60 mg, 0.23 mmol) and 4-nitrobenzoyl chloride (57 mg, 0.30 mmol). The crude product was purified by preparative HPLC (20-100% CH₃CN/MeOH (1:1) in H₂O (0.01% TFA)) which provided after lyophilization 38 mg (42%) of the title compound as a colorless solid; ¹H NMR (400 MHz, MeOD-d4) δ=8.30-8.35 (m, 2H), 8.08-8.13 (m, 2H), 7.78-7.83 (m, 2H), 7.67-7.73 (m, 2H).

### Synthetic Example 23: 3-cyano-N-(4-(1,1,1,3,3,3-hexafluoro-2-hydroxypropan-2-yl)phenyl)benzamide

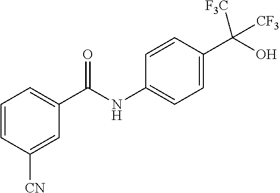 [SMILES: N#Cc1cccc(C(=O)Nc2ccc(C(O)(C(F)(F)F)C(F)(F)F)cc2)c1]

The title compound was prepared according to the Synthetic Example 2 from 4-(1-hydroxy-1-trifluoromethyl-2,2,2-trifluoroethyl)aniline (60 mg, 0.23 mmol) and 3-cyanobenzoyl chloride (51 mg, 0.30 mmol). The crude product was purified by preparative HPLC (20-100% CH₃CN/MeOH (1:1) in H₂O (0.01% TFA)) which provided after lyophilization 80 mg (94%) of the title compound as a colorless solid; ¹H NMR (400 MHz, MeOD-d4) δ=8.31 (s, 1H), 8.20-8.27 (m, 1H), 7.90-7.96 (m, 1H), 7.80-7.86 (m, 2H), 7.66-7.76 (m, 3H).

### Synthetic Example 24: 4-cyano-N-(4-(1,1,1,3,3,3-hexafluoro-2-hydroxypropan-2-yl)phenyl)benzamide

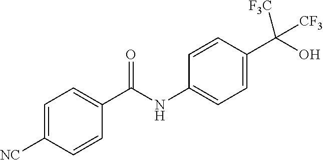 [SMILES: N#Cc1ccc(C(=O)Nc2ccc(C(O)(C(F)(F)F)C(F)(F)F)cc2)cc1]

The title compound was prepared according to the Synthetic Example 2 from 4-(1˜hydroxy-1-trifluoromethyl-2,2,2-trifluoroethyl)aniline (50 mg, 0.19 mmol) and p-toluoyl chloride (38 mg, 0.23 mmol), The crude product was purified by preparative HPLC (20-100% CH₃CN/MeOH (1:1) in H₂O (0.01% TFA)) which provided after lyophilization 49 mg (65%) of the title compound as a colorless solid; ¹H NMR (400 MHz, MeOD-d4) δ=8.06-8.11 (m, 2H), 7.86-7.91 (m, 2H), 7.81-7.86 (m, 2H), 7.71-7.76 (m, 2H).

### Synthetic Example 25: N-(4-(1,1,1,3,3,3-hexafluoro-2-hydroxypropan-2-yl)phenyl)-[1,1-biphenyl]-2-carboxamide

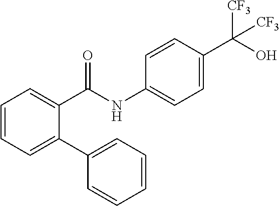 [SMILES: O=C(Nc1ccc(C(O)(C(F)(F)F)C(F)(F)F)cc1)c1ccccc1-c1ccccc1]

In a dried MW flask under Ar were introduced 2-chloro-N-(4-(1,1,1,3,3,3-hexafluoro-2-hydroxypropan-2-yl)phenyl)benzamide (SR0987) (30 mg, 0.075 mmol), phenyl boronic acid (11 mg, 0.091 mmol), K₂CO₃ (52 mg, 0.38 mmol), 0.4 mL of dioxane and 0.08 mL H₂O. The flask was purged three times with Ar before the addition of Pd(dppf)Cl₂ (6 mg, 0.008 mmol). The flask was sealed and the reaction mixture was stirred 5h at 120° C. After removal of the solvent in vacuo, the crude product was purified by preparative HPLC (20-100% CH₃CN/MeOH (1:1) in H₂O (0.01% TFA)) which provided after lyophilization 7 mg (21%) of the title compound as a colorless solid; ¹H NMR (400 MHz, MeOD-d4) δ=8.21 (t, J=1.77 Hz, 1H), 7.91-7.96 (m, 1H), 7.83-7.88 (m, 3H), 7.70-7.76 (m, 4H), 7.58-7.64 (m, 11H), 7.46-7.51 (m, 2H), 7.36-7.41 (m, 1H).

### Synthetic Example 26: N-(4-(1,1,1,3,3,3-hexafluoro-2-hydroxypropan-2-yl)phenyl)-[1,1′-biphenyl]-3-carboxamide

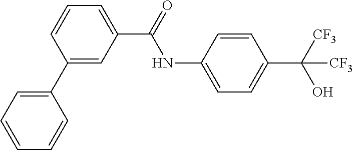 [SMILES: O=C(Nc1ccc(C(O)(C(F)(F)F)C(F)(F)F)cc1)c1cccc(-c2ccccc2)c1]

The title compound was prepared according to the Synthetic Example 2 from 4-(1-hydroxy-1-trifluoromethyl-2,2,2-trifluoroethyl)aniline (50 mg, 0.19 mmol) and biphenyl-3-carbonyl chloride (50 mg, 0.23 mmol). The crude product was purified by column chromatography on silica gel without any workup by hexane/AcOEt (8/2) to obtain 32 mg (38%) of the title compound as a light yellow powder. ¹H NMR (400 MHz, MeOD-d4) δ=8.20 (t, J=1.64 Hz, 1H), 7.91-7.94 (m, 1H), 7.83-7.88 (m, 3H), 7.69-7.76 (m, 4H), 7.57-7.62 (m, 1H), 7.45-7.50 (n, 2H), 7.35-7.41 (m, 1H).

### Synthetic Example 27: N-(4-(1,1,1,3,3,3-hexafluoro-2-hydroxypropan-2-yl)phenyl)-[1,1′-biphenyl]-4-carboxamide

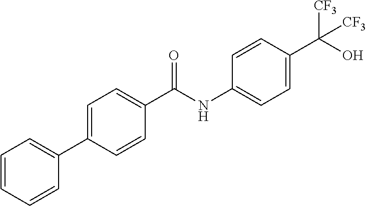 [SMILES: O=C(Nc1ccc(C(O)(C(F)(F)F)C(F)(F)F)cc1)c1ccc(-c2ccccc2)cc1]

The title compound was prepared according to the Synthetic Example 2 from 4-(1-hydroxy-1-trifluoromethyl-2,2,2-trifluoroethyl)aniline (50 mg, 0.19 mmol) and 4-biphenylbenzoyl chloride (50 mg, 0.23 mmol). The crude product was purified by preparative HPLC (20-100% CH₃CN/MeOH (1:1) in H₂O (0.01% TEA)) which provided after lyophilization 80 mg (94%) of the title compound as a colorless solid; ¹H NMR (400 MHz, MeOD-d4) δ=8.01-8.06 (m, 2H), 7.83-7.88 (m, 2H), 7.75-7.80 (m, 2H), 7.71-7.75 (m, 2H), 7.66-7.71 (m, 2H), 7.45-7.50 (m, 2H), 7.40 (d, J=7.45 Hz, 1H).

### Synthetic Example 28: 2,4-dichloro-N-(4-(1,1,1,3,3,3-hexafluoro-2-hydroxypropan-2-yl)phenyl)benzamide

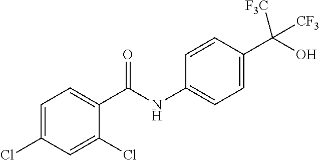 [SMILES: O=C(Nc1ccc(C(O)(C(F)(F)F)C(F)(F)F)cc1)c1ccc(Cl)cc1Cl]

The title compound was prepared according to the Synthetic Example 2 from 4-(1-hydroxy-1-trifluoromethyl-2,2,2-trifluoroethyl)aniline (40 mg, 0.15 mmol) and 2,4-dichlorobenzoyl chloride (26 μL, 0.18 mmol). The crude product was purified by column chromatography on silica gel without any workup by hexane/AcOEt (812) to obtain 63 mg (94%) of the title compound as a white powder; ¹H NMR (400 MHz, MeOD-d4) δ 7.70-7.81 (m, 4H), 7.53-7.63 (m, 2H), 7.45 (dd, J=1.77, 8.34 Hz, 1H).

### Synthetic Example 29: 2,6-dichloro-N-(4-(1,1,1,3,3,3-hexafluoro-2-hydroxypropan-2-yl)phenyl)benzamide

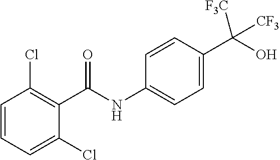 [SMILES: O=C(Nc1ccc(C(O)(C(F)(F)F)C(F)(F)F)cc1)c1c(Cl)cccc1Cl]

The title compound was prepared according to the Synthetic Example 2 from 4-(1-hydroxy-1-trifluoromethyl-2,2,2-trifluoroethyl)aniline (40 my, 0.15 mmol) and 2,6-dichlorobenzoyl chloride (27 μL, 0.18 mmol). The crude product was purified by preparative HPLC (20-100% CH₃CN/MeOH (1:1) in H₂O (0.01% TFA)) which provided after lyophilization 20 mg (30%) of the title compound as a colorless solid; ¹H NMR (400 MHz, CDCl₃) δ=7.74-7.76 (m, 4H), 7.62 (br, s, 1H), 7.30-7.40 (m, 3H).

### Synthetic Example 30: 3,5-dichloro-N-(4-(1,1,1,3,3,3-hexafluoro-2-hydroxypropan-2-yl)phenyl)benzamide

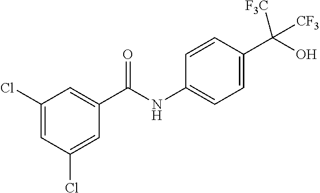 [SMILES: O=C(Nc1ccc(C(O)(C(F)(F)F)C(F)(F)F)cc1)c1cc(Cl)cc(Cl)c1]

The title compound was prepared according to the Synthetic Example 2 from 4-(1-hydroxy-1-trifluoromethyl-2,2,2-trifluoroethyl)aniline (40 mg, 0.15 mmol) and 3,5-dichlorobenzoyl chloride (35 mg, 0.18 mmol). The crude product was purified by column chromatography on silica gel without any workup by hexane/AcOEt (7/3) to obtain 52 mg (78%) of the title compound as a white powder; ¹H NMR (400 MHz, MeOD-d4) δ=7.91 (d, J=1.77 Hz, 2H), 7.79-7.85 (m, 2H), 7.69-7.75 (m, 2H), 7.65 (t, J=1.77 Hz, 1H).

### Synthetic Example 31: 2,3-dichloro-N-(4-(1,1,1,3,3,3-hexafluoro-2-hydroxypropan-2-yl)phenyl)benzamide

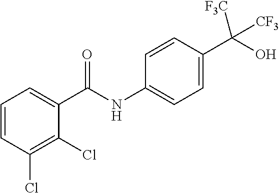 [SMILES: O=C(Nc1ccc(C(O)(C(F)(F)F)C(F)(F)F)cc1)c1cccc(Cl)c1Cl]

The title compound was prepared according to the Synthetic Example 2 from 4-(1-hydroxy-1-trifluoromethyl-2,2,2-trifluoroethyl)aniline (40 mg, 0.15 mmol) and 2,3-dichlorobenzoyl chloride (26 μL, 0.18 mmol). The crude product was purified by preparative HPLC (20-100% CH₃CN/MeOH (1:1) in H₂O (0.01% TFA)) which provided after lyophilization 51 mg (76%) of the title compound as a colorless solid; ¹H NMR (400 MHz, CDCl₃) δ=7.80 (br. s., 1H), 7.74-7.76 (m, 4H), 7.59 (dd, J=7, 78, 11.95 Hz, 1H), 7.59 (dd, J=7.89, 15.13 Hz, 1H), 7.31-7.38 (m, 1H).

### Synthetic Example 32: 5-bromo-2-chloro-N-(4-(1,1,1,3,3,3-hexafluoro-2-hydroxypropan-2-yl)phenyl)benzamide

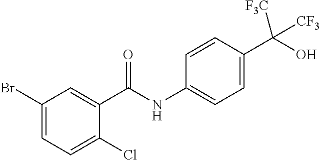 [SMILES: O=C(Nc1ccc(C(O)(C(F)(F)F)C(F)(F)F)cc1)c1cc(Br)ccc1Cl]

The title compound was prepared according to the Synthetic Example 2 from 4-(1-hydroxy-1-trifluoromethyl-2,2,2-trifluoroethyl)aniline (50 mg, 0.19 mmol) and 5-bromo-2-chlorobenzoyl chloride (60 mg, 0.23 mmol), The crude product was purified by column chromatography on silica gel without any workup by hexane/AcOEt (7/3) to obtain 74 mg (80%) of the title compound as a white powder; ¹H NMR (400 MHz, MeOD-d4) δ=7.76-7.81 (m, 2H), 7.70-7.76 (m, 3H), 7.63 (dd, J=2.41, 8.55 Hz, 1H), 7.43 (d, J=8.55 Hz, 1H).

### Synthetic Example 33: N-(4-(1,1,1,3,3,3-hexafluoro-2-hydroxypropan-2-yl)phenyl)furan-2-carboxamide

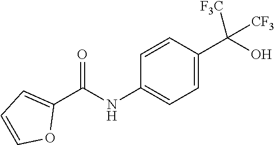 [SMILES: O=C(Nc1ccc(C(O)(C(F)(F)F)C(F)(F)F)cc1)c1ccco1]

The title compound was prepared according to the Synthetic Example 2 from 4-(1-hydroxy-1-trifluoromethyl-2,2,2-trifluoroethyl)aniline (50 mg, 0.19 mmol) and 2-furoyl chloride (23 μL, 0.23 mmol). The crude product was purified by preparative HPLC (20-100% CH₃CN/MeOH (1:1) in H₂O (0.01% TFA)) which provided after lyophilization 29 mg (48%) of the title compound as a colorless solid; ¹H NMR (400 MHz, MeOD-d4) δ 7.79-7.85 (m, 2H), 7.75 (dd, J=0.76, 1.77 Hz, 1H), 7.67-7.73 (m, 2H), 7.29 (dd, J 0.76, 3.54 Hz, 1H), 6.65 (dd, J=1.77, 3.54 Hz, 1H).

### Synthetic Example 34: N-(4-(1,1,1,3,3,3-hexafluoro-2-hydroxypropan-2-yl)phenyl)thiophene-2-carboxamide

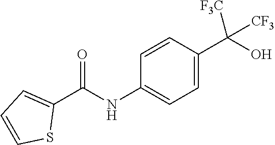 [SMILES: O=C(Nc1ccc(C(O)(C(F)(F)F)C(F)(F)F)cc1)c1cccs1]

The title compound was prepared according to the Synthetic Example 2 from 4-(1-hydroxy-1-trifluoromethyl-2,2,2-trifluoroethyl)aniline (200 mg, 0.77 mmol) and 2-thiophenecarbonyl chloride (116 μL, 1.08 mmol). The crude product was purified by preparative HPLC (20-100% CH₃CN/MeOH (1:1) in H₂O (0.01% TFA)) which provided after lyophilization 260 mg (91%) of the title compound as a colorless solid; ¹H NMR (400 MHz, CDCl₃) δ=7.76 (br. s., 1H), 7.72-7.74 (m, 4H), 7.68 (dd, J=1, 21, 3.84 Hz, 1H), 7.60 (dd, J=1.21, 4.93 Hz, 1H), 7.16 (dd, J=3.84, 4.93 Hz, 1H).

### Synthetic Example 35: N-(4-(1,1,1,3,3,3-hexafluoro-2-hydroxypropan-2-yl)phenyl)picolinamide

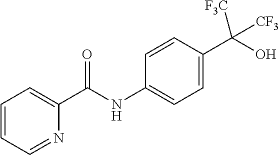 [SMILES: O=C(Nc1ccc(C(O)(C(F)(F)F)C(F)(F)F)cc1)c1ccccn1]

The title compound was prepared according to the Synthetic Example 2 from 4-(1-hydroxy-1-trifluoromethyl-2,2,2-trifluoroethyl)aniline (50 mg, 0.19 mmol) and picolinoyl chloride hydrochloride (41 mg, 0.23 mmol), The crude product was purified by column chromatography on silica gel without any workup by hexane/AcOEt (6/4) to obtain 60 mg (85%) of the title compound as a light yellow powder. ¹H NMR (400 MHz, MeOD-d4) δ=8.65 8.72 (m, 1H), 8.21 (td, J=0.99, 7.89 Hz, 1H), 7.99 (dt, J=1.75, 7.89 Hz, 1H), 7.89-7.95 (m, 2H), 7.69-7.78 (m, J=8.77 Hz, 2H), 7.56-7.60 (m, 1H).

### Synthetic Example 36: N-(4-(1,1,1,3,3,3-hexafluoro-2-hydroxypropan-2-yl)phenyl)nicotinamide

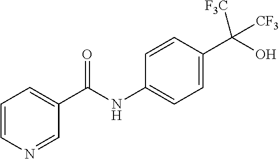 [SMILES: O=C(Nc1ccc(C(O)(C(F)(F)F)C(F)(F)F)cc1)c1cccnc1]

The title compound was prepared according to the Synthetic Example 2 from 4-(1-hydroxy-1-trifluoromethyl-2,2,2-trifluoroethyl)aniline (60 mg, 0.22 mmol) and nicotinoyl chloride hydrochloride (58 mg, 0.32 mmol). The crude product was purified by preparative HPLC (20-100% CH₃CN/MeOH (1:1) in H₂O (0.01% TEA)) which provided after lyophilization 47 mg (56%) of the title compound as a colorless solid as a TFA salt, ¹H NMR (400 MHz, MeOD-d4) δ=9.27 (s, 1H), 8.87-8.97 (m, 1H), 8.84 (d, J=8.11 Hz, 1H), 8.02 (dd, J=5, 48, 8.11 Hz, 1H), 7.81-7.90 (m, 2H), 7.71-7.80 (m, 2H).

### Synthetic Example 37: N-(4-(1,1,1,3,3,3-hexafluoro-2-hydroxypropan-2-yl)phenyl)isonicotinamide

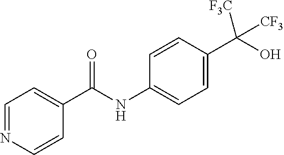 [SMILES: O=C(Nc1ccc(C(O)(C(F)(F)F)C(F)(F)F)cc1)c1ccncc1]

The title compound was prepared according to the Synthetic Example 2 from 4-(1-hydroxy-1-trifluoromethyl-2,2,2-trifluoroethyl)aniline (50 mg, 0.19 mmol) and isonicotinoyl chloride hydrochloride (41 mg, 0.23 mmol). The crude product was purified by preparative HPLC (20-100% CH₃CN/MeOH (1:1) in H₂O (0.01% TFA)) which provided after lyophilization 24 mg (26%) of the title compound as a colorless solid as TFA salt; ¹H NMR (400 MHz, MeOD-d4) δ=8.93 (d, J=4.80 Hz, 2H), 8.21-8.29 (m, 2H), 7.84-7.91 (m, 2H), 7.72-7.79 (m, 2H).

### Synthetic Example 38: N-(4-(1,1,1,3,3,3-hexafluoro-2-hydroxypropan-2-yl)phenyl)-1-naphthamide

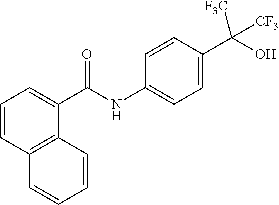 [SMILES: O=C(Nc1ccc(C(O)(C(F)(F)F)C(F)(F)F)cc1)c1cccc2ccccc12]

The title compound was prepared according to the Synthetic Example 2 from 4-(1-hydroxy-1-trifluoromethyl-2,2,2-trifluoroethyl)aniline (60 mg, 0.22 mmol) and 1-naphthoyl chloride (59 mg, 0.30 mmol). The crude product was purified by preparative HPLC (20-100% CH₃CN/MeOH (1:1) in H₂O (0.01% TFA)) which provided after lyophilization 47 mg (52%) of the title compound as a colorless solid; ¹H NMR (400 MHz, MeOD-d4) δ=8.21-8.27 (m, 1H), 8.02 (d, J=8.33 Hz, 1H), 7.92-7.97 (m, 1H), 7.87 (d, J=8.99 Hz, 2H), 7.72-7.78 (m, 3H), 7.52-7.60 (m, 3H).

### Synthetic Example 39: 3-chloro-N-(4-(1,1,1,3,3,3-hexafluoro-2-hydroxypropan-2-yl)phenyl)picolinamide

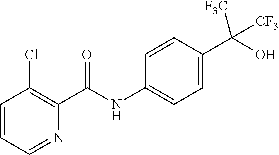 [SMILES: O=C(Nc1ccc(C(O)(C(F)(F)F)C(F)(F)F)cc1)c1ncccc1Cl]

The title compound was prepared according to the Synthetic Example 2 from 4-(1-hydroxy-1-trifluoromethyl-2,2,2-trifluoroethyl)aniline (50 mg, 0.19 mmol) and 3-chloropicolinoyl chloride (40 mg, 0.23 mmol). The crude product was purified by column chromatography on silica gel without any workup by hexane/AcOEt (7/3) to obtain 75 mg (97%) of the title compound as a white powder; ¹H NMR (400 MHz, MeOD-d4) δ=8.57 (dd, J=1.32, 4.60 Hz, 1H), 7.96-8.03 (m, 1H), 7.81-7.89 (m, 2H), 7.70-7.77 (m, 2H), 7.53 (dd, J=4.60, 8.22 Hz, 1H).

### Synthetic Example 40: 2-chloro-N-(4-(1,1,1,3,3,3-hexafluoro-2-hydroxypropan-2-yl)-2-methylphenyl)benzamide

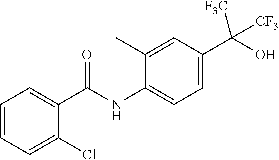 [SMILES: Cc1cc(C(O)(C(F)(F)F)C(F)(F)F)ccc1NC(=O)c1ccccc1Cl]

The title compound was prepared according to the Synthetic Example 2 from 2-(4-amino-3-methylphenyl)-1,1,1,3,3,3-hexafluoropropan-2-ol (100 mg, 0.37 mmol) and 2-chlorobenzoyl chloride (56 μL, 0.44 mmol). The crude product was purified by column chromatography on silica gel without any workup by hexane/AcOEt (7/3) to obtain 122 mg (81%) of the title compound as a white powder; ¹H NMR (400 MHz, MeOD-d4) δ=7.68 (s, 1H), 7.58-7.65 (m, 3H), 7.50-7.55 (m, 1H), 7.39-7.50 (m, 2H), 2.41 (s, 3H).

### Synthetic Example 41: 2-chloro-N-(2-chloro-4-(2-chloro-1,1,1,3,3,3-hexafluoropropan-2-yl)phenyl)benzamide

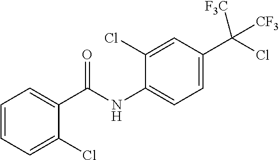 [SMILES: O=C(Nc1ccc(C(Cl)(C(F)(F)F)C(F)(F)F)cc1Cl)c1ccccc1Cl]

The title compound was prepared according to the Synthetic Example 2 from 2-(4-amino-3-chlorophenyl)-1,1,1,3,3,3-hexafluoropropan-2-ol (50 mg, 0.17 mmol) and 2-chlorobenzoyl chloride (26 μL, 0.20 mmol). The crude product was purified by column chromatography on silica gel without any workup by hexane/AcOEt (7/3) to obtain 51 mg (69%) of the title compound as a white powder; ¹H NMR (400 MHz, MeOD-d4) δ=8.08 (d, J=8.59 Hz, 1H), 7.86 (d, J=1.77 Hz, 1H), 7.63-7.78 (m, 2H), 7.39-7.57 (m, 3H).

### Synthetic Example 42: 2-chloro-N-(4-(2-chloro-1,1,1,3,3,3-hexafluoropropan-2-yl)-2-methoxyphenyl)benzamide

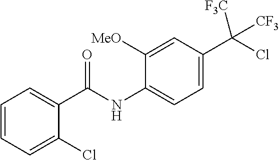 [SMILES: COc1cc(C(Cl)(C(F)(F)F)C(F)(F)F)ccc1NC(=O)c1ccccc1Cl]

The title compound was prepared according to the Synthetic Example 2 from 2-(4-amino-3-methoxyphenyl)-1,1,1,3,3,3-hexafluoropropan-2-ol (100 mg, 0.35 mmol) and 2-chlorobenzoyl chloride (53 μL, 0.41 mmol). The crude product was purified by column chromatography on silica gel without any workup by hexane/AcOEt (713) to obtain 105 mg (71%) of the title compound as a white powder; ¹H NMR (400 MHz, MeOD-d4)=8.27 (d, J=8.59 Hz, 1H), 7.63 (d, J=7.33 Hz, 1H), 7.39-7.55 (m, 4H), 7.35 (d, J=8.59 Hz, 1H), 3.90 (s, 3H).

### Synthetic Example 43: 2-chloro-N-(4-(1,1,1,3,3,3-hexafluoro-2-hydroxypropan-2-yl)-3-methylphenyl)benzamide

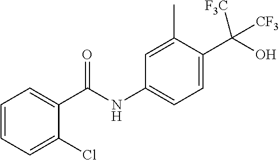 [SMILES: Cc1cc(NC(=O)c2ccccc2Cl)ccc1C(O)(C(F)(F)F)C(F)(F)F]

The title compound was prepared according to the Synthetic Example 2 from 2-(4-amino-2-methylphenyl)-1,1,1,3,3,3-hexafluoropropan-2-ol (100 mg, 0.37 mmol) and 2-chlorobenzoyl chloride (55 μL, 0.44 mmol). The crude product was purified by preparative HPLC (20-100% CH₃CN/MeOH (1:1) in H₂O (0.01% TFA)) which provided after lyophilization 101 mg (67%) of the title compound as a colorless solid; ¹H NMR (400 MHz, MeOD-d4) δ=8.35 (s, 1H), 7.62 (dd, J=1.64, 7.34 Hz, 1H), 7.42-7.56 (m, 4H), 7.07-7.10 (m, 1H), 2.41 (s, 3H).

### Synthetic Example 44: 2-chloro-N-(3-chloro-4-(1,1,1,3,3,3-hexafluoro-2-hydroxypropan-2-yl)phenyl)benzamide

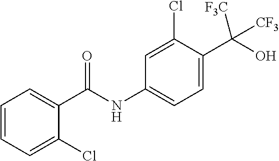 [SMILES: O=C(Nc1ccc(C(O)(C(F)(F)F)C(F)(F)F)c(Cl)c1)c1ccccc1Cl]

The title compound was prepared according to the Synthetic Example 2 from 2-(4-amino-2-chlorophenyl)-1,1,1,3,3,3-hexafluoropropan-2-ol (100 mg, 0.34 mmol) and 2-chlorobenzoyl chloride (52 μL, 0.41 mmol). The crude product was purified by column chromatography on silica gel without any workup by hexane/AcOEt (7/3) to obtain 103 mg (70%) of the title compound as a white powder; ¹H NMR (400 MHz, MeOD-d4) δ=8.76 (d, J 2.02 Hz, 1H), 7.63-7.73 (m, 1H), 7.36-7.57 (m, 4H), 7.07-7.17 (m, 1H).

### Synthetic Example 45: 2-chloro-N-(4-(1,1,1,3,3,3-hexafluoro-2-hydroxypropan-2-yl)-3-methoxyphenyl)benzamide

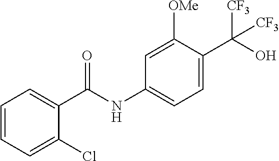 [SMILES: COc1cc(NC(=O)c2ccccc2Cl)ccc1C(O)(C(F)(F)F)C(F)(F)F]

The title compound was prepared according to the Synthetic Example 2 from 2-(4-amino-2-methoxyphenyl)-1,1,1,3,3,3-hexafluoropropan-2-ol (100 mg, 0.35 mmol) and 2-chlorobenzoyl chloride (53 μL, 0.41 mmol). The crude product was purified by preparative HPLC (20-100% CH₃CN/MeOH (1:1) in H₂O (0.01% TFA)) which provided after lyophilization 42 mg (28%) of the title compound as a colorless solid; ¹H NMR (400 MHz, MeOD-d4) δ=7.71 (d, J=2.19 Hz, 1H), 7.66 (d, J=8.77 Hz, 1H), 7.55-7.59 (m, 1H), 7.40-7.53 (m, 3H), 7.28 (dd, J=2.19, 8.77 Hz, 1H), 3.91 (s, 3H).

### Synthetic Example 46: 2-chloro-N-(4-(1,1,1,3,3,3-hexafluoro-2-methoxypropan-2-yl)phenyl)benzamide

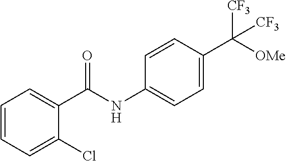 [SMILES: COC(c1ccc(NC(=O)c2ccccc2Cl)cc1)(C(F)(F)F)C(F)(F)F]

The title compound was prepared according to the Synthetic Example 2 from 4-(1,1,1,3,3,3-hexafluoro-2-methoxypropan-2-yl)aniline (50 mg, 0.18 mmol) and 2-chlorobenzoyl chloride (28 μL, 0.22 mmol). The crude product was purified by column chromatography on silica gel without any workup by hexane/AcOEt (7/3) to obtain 63 mg (84%) of the title compound as a white powder; ¹H NMR (400 MHz, MeOD-d4) δ=7.79-7.97 (m, 2H), 7.55-7.64 (m, 3H), 7.40-7.54 (m, 3H), 3.50 (s, 3H).

### Synthetic Example 47: 2-chloro-N-(4-(perfluoropropan-2-yl)phenyl)benzamide

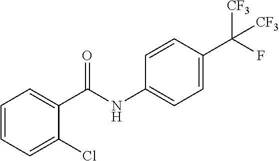 [SMILES: O=C(Nc1ccc(C(F)(C(F)(F)F)C(F)(F)F)cc1)c1ccccc1Cl]

In a dried MW flask under Ar were introduced 2-chloro-N-(4-(1,1,1,3,3,3-hexafluoro-2-hydroxypropan-2-yl)phenyl)benzamide (SR0987) (50 mg, 0.13 mmol), Deoxo-Fluor® (70 μL, 0.38 mmol) and 0.4 mL of anhydrous DCM. The flask was sealed and the reaction mixture was stirred overnight at 50° C.; The crude product was purified by preparative HPLC (20-100% CH₃CN/MeOH (1:1) in H₂O (0.01% TFA)) which provided after lyophilization 24 mg (48%) of the title compound as a colorless solid: ¹H NMR (400 MHz, DMSO-d6) δ=10.90 (s, 1H), 7.93-8.00 (m, 2H), 7.64-7.72 (m, 2H), 7.58-7.63 (m, 2H), 7.51-7.55 (m, 1H), 7.45-7.50 (m, 1H).

### Synthetic Example 48: 4-(2-chlorobenzamido)benzoic acid

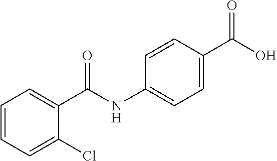 [SMILES: O=C(O)c1ccc(NC(=O)c2ccccc2Cl)cc1]

To the solution of 4-aminobenzoic acid (47 mg, 0.34 mmol) in 2 ml of anhydrous THE was introduced 2-chlorobenzoyl chloride (36 μL, 0.29 mmol). The obtained solution was cooled at 0° C. followed by the addition dropwise of Et₃N (60 μL, 0.43 mmol). After 4h at room temperature, iced cooled HCl 1N was added. The obtained solid was filtered and purified by preparative HPLC (20-100% CH₃CN/MeOH (1:1) in H₂O (0.01% TEA)) which provided after lyophilization 36 mg (46%) of the title compound as a colorless solid: ¹H NMR (400 MHz, MeOD-d4) δ=8.00-8.06 (m, 2H), 7.77-7.83 (m, 2H), 7.55-7.60 (m, 1H), 7.41-7.55 (m, 3H).

### Synthetic Example 49: N-(4-(tert-butyl)phenyl)-2-chlorobenzamide

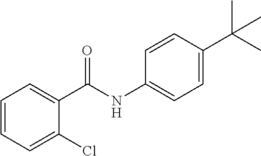 [SMILES: CC(C)(C)c1ccc(NC(=O)c2ccccc2Cl)cc1]

The title compound was prepared according to the Synthetic Example 2 from 4-tert-butylaniline (43 μL, 0.27 mmol) and 2-chlorobenzoyl chloride (41 μL, 0.32 mmol). The crude product was purified by column chromatography on silica gel without any workup by hexane/AcOEt (7/3) to obtain 66 mg (86%) of the title compound as a white powder; ¹H NMR (400 MHz, MeOD-d4) δ=7.57-7.62 (m, 2H), 7.51-7.55 (m, 1H), 7.45-7.51 (m, 1H), 7.37-7.45 (m, 4H), 1.33 (s, 9H).

### Synthetic Example 50: N-(4-benzamidophenyl)-2-chlorobenzamide

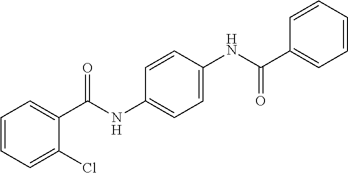 [SMILES: O=C(Nc1ccc(NC(=O)c2ccccc2Cl)cc1)c1ccccc1]

The title compound was prepared according to the Synthetic Example 2 from N-(4-aminophenyl)benzamide (40 mg, 0.19 mmol) and 2-chlorobenzoyl chloride (29 μL, 0.23 mmol). The crude product was purified by column chromatography on silica gel without any workup by hexane/AcOEt (7/3) to obtain 62 mg (94%) of the title compound as a white powder; ¹H NMR (400 MHz, MeOD-d4) δ=7.91-7.97 (m, 2H), 7.67-7.74 (m, 4H), 7.41-7.61 (m, 7H).

### Synthetic Example 51: 2-chloro-N-(4-(phenylsulfonamido)phenyl)benzamide

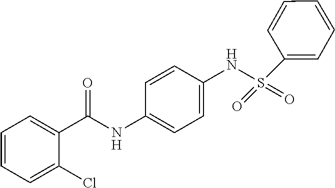 [SMILES: O=C(Nc1ccc(NS(=O)(=O)c2ccccc2)cc1)c1ccccc1Cl]

The title compound was prepared according to the Synthetic Example 2 from N-(4-aminophenyl)benzenesulfonamide (50 mg, 0.20 mmol) and 2-chlorobenzoyl chloride (31 μL, 0.24 mmol). The crude product was purified by column chromatography on silica gel without any workup by hexane/AcOEt (713) to obtain 51 mg (65%) of the title compound as a white powder; ¹H NMR (400 MHz, MeOD-d4) δ=7.72-7.78 (m, 2H), 7.37-7.60 (m, 9H), 7.05-7.11 (m, 2H).

### Synthetic Example 52: 2-chloro-N-(4-(1,1,1,3,3,3-hexafluoro-2-hydroxypropan-2-yl)phenyl)benzenesulfonamide

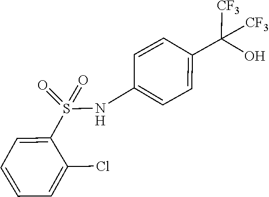 [SMILES: O=S(=O)(Nc1ccc(C(O)(C(F)(F)F)C(F)(F)F)cc1)c1ccccc1Cl]

To the solution of 4-(1-hydroxy-1-trifluoromethyl-2,2,2-trifluoroethyl)aniline (40 mg, 0.15 mmol) in 250 μL of pyridine was added 2-chlorobenzenesulfonyl chloride (25 μL, 0.18 mmol). The reaction was stirred overnight at 80° C. Removal of the solvent provided the crude product, which was purified by preparative HPLC (20-100% CH₃CN/MeOH (1:1) in H₂O (0.01% TFA)) which provided after lyophilization 58 mg (87%) of the title compound as a colorless solid; ¹H NMR (400 MHz, CDCl₃) δ=8.07-8.10 (m, 1H), 7.54-7.61 (m, 2H), 7.47-7.54 (m, 2H), 7.36-7.43 (m, 1H), 7.26 (br. s., 11H), 7.16-7.22 (m, 2H).

### Synthetic Example 53: 2-(4-((2-chlorobenzyl)amino)phenyl)-1,1,1,3,3,3-hexafluoropropan-2-ol

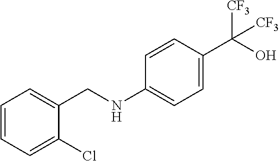 [SMILES: OC(c1ccc(NCc2ccccc2Cl)cc1)(C(F)(F)F)C(F)(F)F]

To the solution of 4-(1-hydroxy-1-trifluoromethyl-2,2,2-trifluoroethyl)aniline (80 mg, 0.31 mmol) in 3 mL of MeOH were added 2-chlorobenzaldehyde (104 μL, 0.93 mmol) and acetic acid (88 μL, 1.54 mmol). The reaction mixture was stirred 30 min followed by the addition of NaBH₃CN (97 mg, 1.54 mmol). The reaction was stirred overnight at 60° C., Removal of the solvent provided the crude product, which was purified by preparative HPLC (20-100% CH₃CN/MeOH (1:1) in H₂O (0.01% TFA)) which provided after lyophilization 95 mg (75%) of the title compound as a colorless solid as TFA salt: ¹H NMR (400 MHz, MeOD-d4) δ=7.30-7.53 (m, 4H), 7.11-7.28 (m, 2H), 6.57-6.73 (m, 2H), 4.42 (s, 2H).

### Synthetic Example 54: N-(2-chlorophenyl)-4-(1,1,1,3,3,3-hexafluoro-2-hydroxypropan-2-yl)benzamide

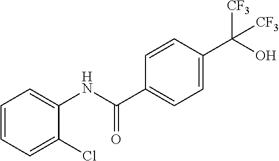 [SMILES: O=C(Nc1ccccc1Cl)c1ccc(C(O)(C(F)(F)F)C(F)(F)F)cc1]

To the solution of 4-(1,1,1,3,3,3-hexafluoro-2-hydroxypropan-2-yl)benzoic acid (50 mg, 0.17 mmol) in 3 mL of anhydrous DCM were added oxalyl chloride (22 μL, 0.26 mmol) and DMF (4 μL, 0.05 mmcl). The reaction was stirred at room temperature for 2h. Removal of the solvent provided 4-(1,1,1,3,3,3-hexafluoro-2-hydroxypropan-2-yl)benzoyl chloride.

The title compound was prepared according to the Synthetic Example 2 from 2-chloroaniline (19 μL, 0.18 mmol) and 4-(1,1,1,3,3,3-hexafluoro-2-hydroxypropan-2-yl)benzoyl chloride. The crude product was purified by preparative HPLC (20-100% CH₃CN/MeOH (1:1) in H₂O (0.01% TFA)) which provided after lyophilization 37 mg (54%) of the title compound as a colorless solid: ¹H NMR (400 MHz, MeOD-d4) δ=8.03-8.14 (m, J=8.55 Hz, 2H), 7.88-7.96 (m, J=8.11 Hz, 2H), 7.73-7.75 (m, 1H), 7.50-7.57 (m, 1H), 7.34-7.43 (m, 1H), 7.26-7.34 (m, 1H).

### Synthetic Example 55: 2-chloro-N-(4-(1,1,1,3,3,3-hexafluoro-2-hydroxypropan-2-yl)phenyl)-N-methylbenzamide

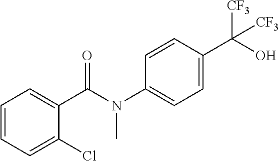 [SMILES: CN(C(=O)c1ccccc1Cl)c1ccc(C(O)(C(F)(F)F)C(F)(F)F)cc1]

The title compound was prepared according to the Synthetic Example 2 from 1,1,1,3,3,3-hexafluoro-2-(4-(methylamino)phenyl)propan-2-ol (100 mg, 0.37 mmol) and 2-chlorobenzoyl chloride (56 μL, 0.44 mmol). The crude product was purified by preparative HPLC (20-100% CH₃CN/MeOH (1:1) in H₂O (0.01% TFA)) which provided after lyophilization 55 mg (36%) of the title compound as a colorless solid; ¹H NMR (400 MHz, MeOD-d4) δ=7.50-7.59 (m, 2H), 7.32-7.34 (m, 2H), 7.11-7.29 (m, 4H), 3.51 (s, 3H)

HDX-MS. Solution-phase amide HDX was performed with a fully automated system as described previously with minor modifications^{30,31}. For differential HDX experiments, 5 μL of a 10 μM RORγt LBD solution (Apo or in complex with 10-excess compound) was diluted to 25 μL with D20-containing HDX buffer, and incubated at 4° C. for; 10 s, 30s, 60s, 900s, and 3,600s, Following on-exchange, unwanted forward or back exchange is minimized and the protein is denatured by dilution to 50 μL with 0.1% TFA in 3M urea (held at 4° C., pH 2.5). Samples are then passed across an immobilized pepsin column (prepared in house) at 50 μL min−1 (0.1% TFA, 15° C.) and the resulting peptides are trapped onto a Ce trap cartridge (Thermo Fisher, Hypersil Gold). Peptides were then gradient eluted (4% CH₃CN to 40% CH₃CN, 0,3% formic acid over 5 minutes, 4° C.) across a 1 mm×50 mm Cia HPLC column (Hypersil Gold, Thermo Fisher) and electrosprayed directly into a high resolution orbitrap mass spectrometer (Exactive, Thermo Fisher). Percent deuterium exchange values for peptide isotopic envelopes at each time point were calculated and processed using HDX Workbench³² and overlaid onto RORγt crystal structures using pyMOL (DeLano Scientific). HDX data is presented as an average of three individual replicates across 6 time points (10s, 60s, 300s, 900s, and 3600s).

NR box peptide interaction assay. A TR-FRET-based interaction assay was used. The His-Sumo RORγ ligand binding domain (LBD) and FITC-labeled SRC1-3 peptide (sequence: ASNLGLEDIIRKALMGSFD) (SEQ ID NO:7) was used. TR-FRET reaction contains 2.5 nM RORγ LBD, 450 nM SRC1-3 peptide in assay buffer (TR-FRET Coregulator Buffer D, Lifetechnologies). The mixtures were incubated for 2 hr at R.T., and fluorescence intensity was measured on an Envision pate reader with excitation at 340 nm and emission at 490 nm and 520 nm. The ratio of intensity at 520 nm/490 nm was used to calculate cofactor recruitment activity.

Luciferase reporter assay. HEK 293T cells were transfected with a UAS: luciferase reporter and a Gal4-RORγ encoding plasmid (using X-trememGENE 9, Roche). Ursolic acid was pretreated before compounds were added. Luciferase activity was measured 20 hr after compound addition.

Gene expression and Cell sorting. Jurkat T cells were pre-incubated with compounds for 48 hr and activated with phorbol 12-myristate 13-acetate (PMA, 50 ng/mL; Sigma) and ionomycin (1 μg/mL; Sigma) for 5 hr. For qPCR, mRNA was isolated with an RNeasy midi kit using DNase I (Qiagen), and cDNA was synthesized with high capacity cDNA Reverse Transcription kit (Applied Biosystems). IL17A, PD-1, and granzyme B gene expression were normalized to the expression of GAPDH. The sequence of primers used in this study are found in Table 2. For cell sorting, activated Jurkat T cells were stained with APC conjugated anti-human PD-1 antibody (eBioscience). Cell sorting was performed using LSRII (BD Bioscience).

T_{H}17 cell differentiation. For naïve T cell differentiation, CD4⁺T cells were enriched by negative selection using a magnetic-activated cell sorter kit (Millipore). Enriched CD4+ T cells activated with 5 μg/mL of plate-abound anti CD3 antibody and 1 μg/mL of anti-CD28 antibody in the presence of 20 μg/mL of anti-IFNγ, 20 μg/mL of anti-IL-4, 1 ng/mL of TGFβ, AND 10 ng/mL of IL-6. Four-five days post differentiation, all cells were stimulated for 5 hr with 5 ng/mL of phorbol-12-myristate-13-acetate (Sigma) and 500 ng/mL of ionomycin (Sigma) contained with brefeldin A solution (eBioscience).

Data analysis and statistics. All experiments were done with three or more biological replicates. Error bars represent standard deviation. Statistics were calculated using an unpaired, two sample Student's t-test.

### DOCUMENTS CITED

1 Huang, Z., Xie, H., Wang, R. & Sun, Z. Retinoid-related orphan receptor gamma t is a potential therapeutic target for controlling inflammatory autoimmunity. Expert opinion on therapeutic targets 11, 737-743, doi:10.1517114728222.11.6.737 (2007).
2 Wang, Y. et at Modulation of retinoic acid receptor-related orphan receptor alpha and gamma activity by 7-oxygenated sterol ligands. The Journal of biological chemistry 285, 5013-5025, doi:10.10741jbc.M109.080614 (2010).
3 Hu, X. et al. Sterol metabolism controls T(H)17 differentiation by generating endogenous RORgamma agonists. Nature chemical biology 11, 141-147, doi:10.1038/nchembio.1714 (2015).
4 Santori, F. R. et al. Identification of natural RORgamma ligands that regulate the development of lymphoid cells. Cell metabolism 21, 286-297, doi:10.1016/,j.cmet.2015.01.004 (2015).
5 Sun, Z. et al Requirement for RORgamma in thymocyte survival and lymphoid organ development. Science 288, 2369-2373 (2000).
6 Skepner, J. et al Pharmacologic inhibition of RORgammat regulates Th17 signature gene expression and suppresses cutaneous inflammation in vivo. J Immunol 192, 2564-2575, doi:10.4049/jimmunol.1302190 (2014),
7 Kryczek, L et al Phenotype, distribution, generation, and functional and clinical relevance of Th17 cells in the human tumor environments. Blood 114, 1141-1149, doi:10.1182/blood-2009-03-208249 (2009).
8 Yen, H. R. st al, Tc17 CD8 T cells: functional plasticity and subset diversity. J Immunol 183, 7161-7168. doi:10.4049/jimmunol.0900368 (2009).
9 Twyman-Saint Victor, C. et al. Radiation and dual checkpoint blockade activate non-redundant immune mechanisms in cancer. Nature 520, 373-377, doi:10.1038/nature14292 (2015).
10 Gubin, M. M. et al. Checkpoint blockade cancer immunotherapy targets tumour-specific mutant antigens. Nature 515, 577-581, doi:10.1038/nature13988 (2014).
11 Zou, W. & Restifo, N. P. T(H)17 cells in tumour immunity and immunotherapy. Nature reviews. Immunology 10, 248-256, doi:10.1038/nri2742 (2010),
12 Topalian, S. L. et at. Safety, activity, and immune correlates of anti-PD-1 antibody in cancer. The New England journal of medicine 366, 2443-2454, doi:10.1056/NEJMoa1200690 (2012).
13 Brahmer, J. R. et al. Safety and activity of anti-PD-L1 antibody in patients with advanced cancer. The New England journal of medicine 366, 2455-2465, doi:A01056/NEJMoa1200694 (2012).
14 Hamid, O. et al. Safety and tumor responses with lambrolizumab (anti-PD-1) in melanoma. The New England journal of medicine 369, 134-144, doi:10.1056/NEJMoa1305133 (2013).
15 Wolchok, J. D. et at. Nivolumab plus ipilimumab in advanced melanoma. The New England journal of medicine 369, 122-133, doi:10.1056/NEJMoa1302369 (2013).
16 Topalian, S. L. et at Survival, durable tumor remission, and long-term safety in patients with advanced melanoma receiving nivolumab. Journal of clinical oncology: official journal of the American Society of Clinical Oncology 32, 1020-1030, doi:10.1200/JCO.2013.53.0105 (2014).
17 Zhang, W. et al. Increasing human Th17 differentiation through activation of orphan nuclear receptor retinoid acid-related orphan receptor gamma (RORgamma) by a class of aryl amide compounds. Molecular pharmacology 82, 583-590, doi:10.1124/mol.112.078667 (2012).
18 Wang, Y. et al. Identification of SR1078, a synthetic agonist for the orphan nuclear receptors RORalpha and RORgamma. ACS chemical biology 5, 1029-1034, doi:10.1021/cb100223d (2010).
19 Rene, O, et al. Minor Structural Change to Tertiary Sulfonamide RORc Ligands Led to Opposite Mechanisms of Action. ACS medicinal chemistry letters 6, 276-281, doi:10.102/ml500420y (2015).
20 Kumar, N. et al. Identification of SR2211: a potent synthetic RORgamma-selective modulator. ACS chemical biology 7, 672-677, doi:10.1021/cb200496y (2012).
21 Chang, M. R., Lyda, B., Kamenecka, T. M. & Griffin, P. R. Pharmacologic repression of retinoic acid receptor-related orphan nuclear receptor gamma is therapeutic in the collagen-induced arthritis experimental model. Arthritis Rheumatol 66, 579-588, doi:10.1002/art.38272 (2014).
22 Chemnitz, J. M., Parry, R. V., Nichols, K. E., June, C. H. & Riley, J. L. SHP-1 and SHP-2 associate with immunoreceptor tyrosine-based switch motif of programmed death 1 upon primary human T cell stimulation, but only receptor ligation prevents T cell activation. J Immunol 173, 945-954 (2004).
23 Nishimura, H., Honjo, T. & Minato, N. Facilitation of beta selection and modification of positive selection in the thymus of PD-1-deficient mice. The Journal of experimental medicine 191, 891-898 (2000).
24 Nishimura, H. et at. Autoimmune dilated cardiomyopathy in PD-1 receptor-deficient mice. Science 291, 319-322, doi:10.1126/science.291.5502.319 (2001).
25 Tarrio, M. L., Grabie, N., Bu, D. X., Sharpe, A. H. & Lichtman, A. H. PD-1 protects against inflammation and myocyte damage in T cell-mediated myocarditis. J Immunol 188, 4876-4884, doi:10.4049/jimmunol.1200389 (2012).
26 Zhang, J. et at Hydrogen/deuterium exchange reveals distinct agonist/partial agonist receptor dynamics within vitamin D receptor/retinoid X receptor heterodimer. Structure 18, 1332-1341, doi:10.1016/j.str.2010.07.007 (2010).
27 Solt, L. A. et at Suppression of TH17 differentiation and autoimmunity by a synthetic ROR ligand, Nature 472, 491-494, doi:10.1038/nature10075 (2011).
28 Marciano, D. P. et at Pharmacological repression of PPARgamma promotes osteogenesis. Nature communications 6, 7443, doi:10,1038/ncomms8443 (2015).
29 Jin, L. et at Structural basis for hydroxycholesterols as natural ligands of orphan nuclear receptor RORgamma. Mol Endocrinol 24, 923-929, doi:10.1210/me.2009-0507 (2010).
30 Goswami, D. et a/. Influence of domain interactions on conformational mobility of the progesterone receptor detected by hydrogen/deuterium exchange mass spectrometry. Structure 22, 961-973, doi:10.1016/j.str.2014.04.013 (2014).
31 West, G. M. at al. Protein conformation ensembles monitored by HDX reveal a structural rationale for abscisic acid signaling protein affinities and activities. Structure 21, 229-235, doi:1.1016/j.str.2012.12.001 (2013).
32 Pascal, B. D, et al. HDX workbench: software for the analysis of HID exchange MS data. J Am Soc Mass Spectrom 23, 1512-1521, doi:10.1007/s13361-012-0419-6 (2012).

All patents and publications referred to herein are incorporated by reference herein to the same extent as if each individual publication was specifically and individually indicated to be incorporated by reference in its entirety.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for enhancing immunity in a patient, comprising administering to the patient an effective amount of an agonist of RORγt comprising a compound of formula (I) 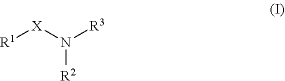
wherein X is C(O) or S(O)₂;
R¹ is phenyl, mono- or independently multi-substituted with J¹;
R² is H or alkyl, wherein any alkyl is optionally mono- or independently multi-substituted with J²;
R³ is phenyl wherein R³ substituted with J³ comprises 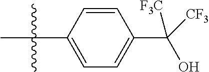 [SMILES: *c1ccc(C(O)(C(F)(F)F)C(F)(F)F)cc1]
or an alkyl, aryl, or arylalkyl ester of the hydroxyl group thereof, or an alkyl, aryl, or arylalkyl ether of the hydroxyl group thereof, wherein a wavy line indicates a point of attachment of J³-substituted R³ to the nitrogen atom bearing R³;
J¹ when present is halo, cyano, nitro, alkoxy, or haloalkoxy; unsubstituted or substituted alkyl, haloalkyl, alkyicarboxamido, arylcarboxamido, or alkoxycarbonyl; unsubstituted or substituted aryl; unsubstituted or substituted arylsulfonyl; unsubstituted or substituted heteroaryl; unsubstituted or substituted heteroarylsulfonyl; or unsubstituted or substituted arylsulfonamido;
J² when present is halo, cyano, nitro, alkoxy, or haloalkoxy; unsubstituted or substituted alkyl, haloalkyl, alkylcarboxamido, arylcarboxamido or alkoxycarbonyl; unsubstituted or substituted aryl; unsubstituted or substituted arylsulfonyl; unsubstituted or substituted heteroaryl; unsubstituted or substituted heteroarylsulfonyl; or unsubstituted or substituted arylsulfonamido;
including any stereoisomer thereof, or any salt, solvate, hydrate, metabolite, or prodrug thereof.

2. The method of claim 1, wherein administration of an effective amount of a compound of formula (Q) increases production of IL17 in situ.

3. A method of treating cancer, comprising administering to a patient afflicted therewith an effective amount of an agonist of RORγt comprising a compound of a compound of formula (I) 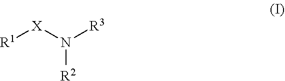
wherein X is C(O) or S(O)₂;
R¹ is phenyl, mono- or independently multi-substituted with J¹;
R² is H or alkyl, wherein any alkyl is optionally mono- or independently multi-substituted with J²;
R³ is phenyl wherein R³ substituted with J³ comprises 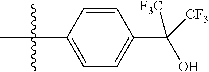 [SMILES: *c1ccc(C(O)(C(F)(F)F)C(F)(F)F)cc1]
or an alkyl, aryl, or arylalkyl ester of the hydroxyl group thereof, or an alkyl, aryl, or arylalkyl ether of the hydroxyl group thereof, wherein a wavy line indicates a point of attachment of J³-substituted R³ to the nitrogen atom bearing R³;
J¹ when present is halo, cyano, nitro, alkoxy, or haloalkoxy; unsubstituted or substituted alkyl, haloalkyl, alkylcarboxamido, arylcarboxamido, or alkoxycarbonyl; unsubstituted or substituted aryl; unsubstituted or substituted arylsulfonyl; unsubstituted or substituted heteroaryl; unsubstituted or substituted heteroarylsulfonyl; or unsubstituted or substituted arylsulfonamido;
J² when present is halo, cyano, nitro, alkoxy, or haloalkoxy; unsubstituted or substituted alkyl, haloalkyl, alkylcarboxamido, arylcarboxamido or alkoxycarbonyl; unsubstituted or substituted aryl; unsubstituted or substituted arylsulfonyl; unsubstituted or substituted heteroaryl; unsubstituted or substituted heteroarylsulfonyl; or unsubstituted or substituted arylsulfonamido;
including any stereoisomer thereof, or any salt, solvate, hydrate, metabolite, or prodrug thereof.

4. The method of claim 1 or 3, wherein the compound of formula (I) is SR0987 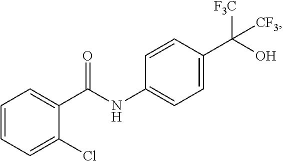 [SMILES: O=C(Nc1ccc(C(O)(C(F)(F)F)C(F)(F)F)cc1)c1ccccc1Cl]
or a pharmaceutically acceptable salt thereof.

5. The method of claim 1 or 3, wherein the compound of formula (I) is any one of: 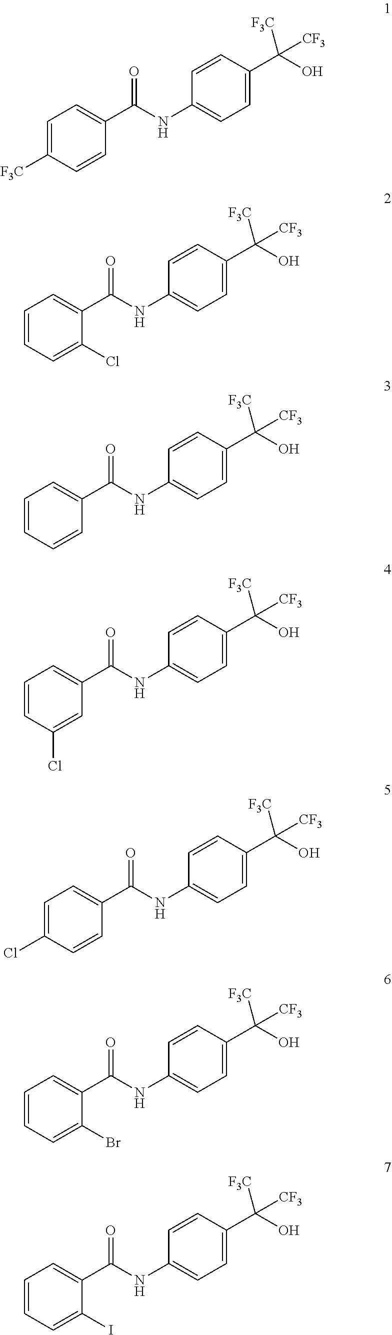 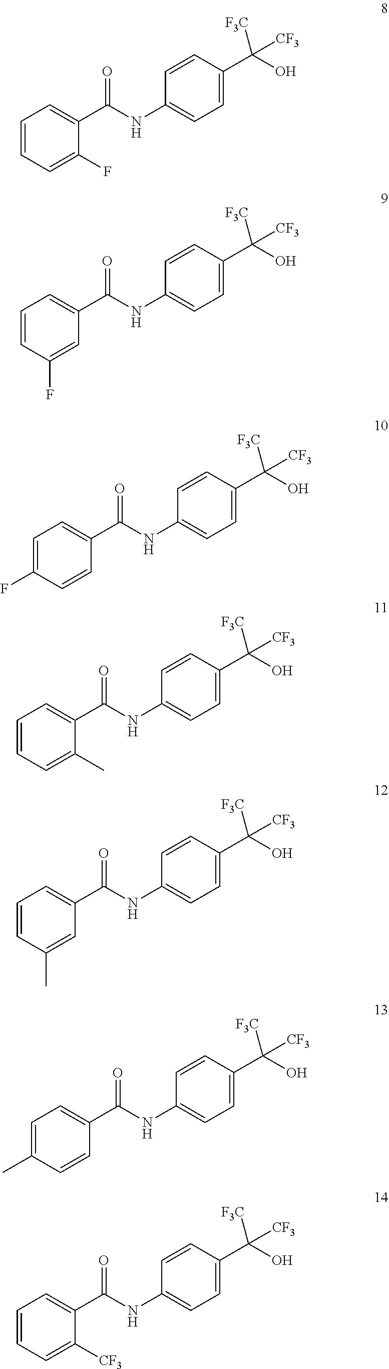 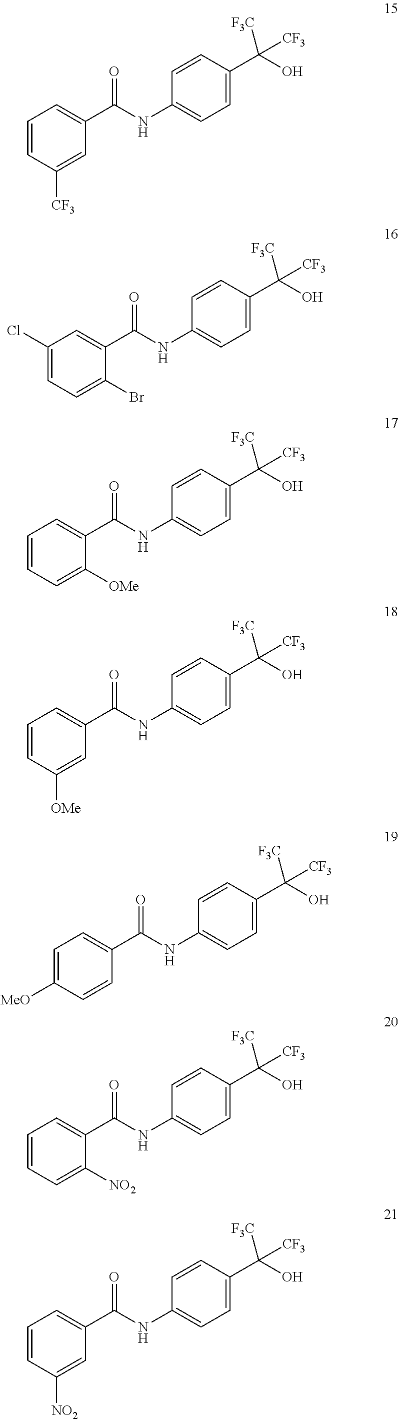 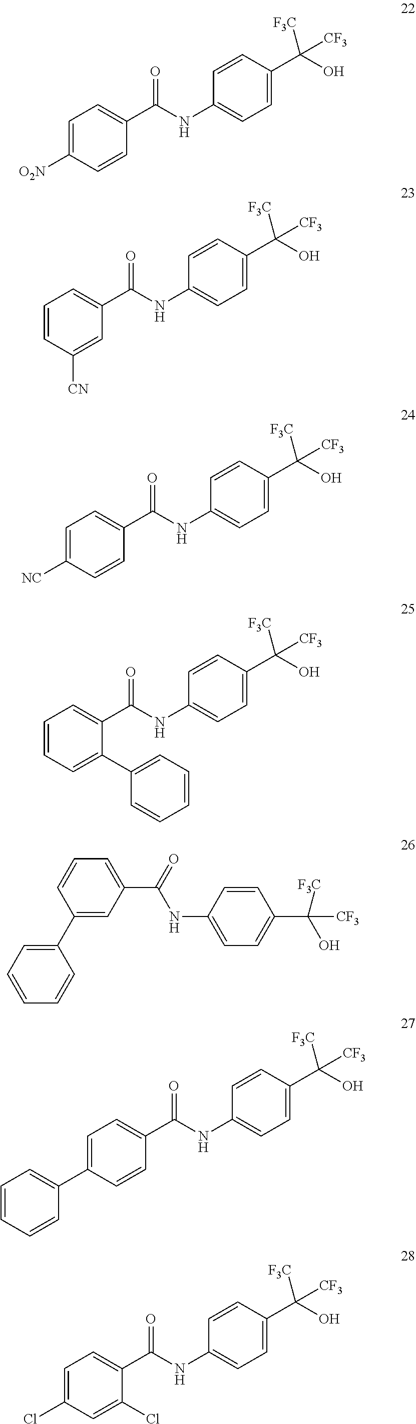 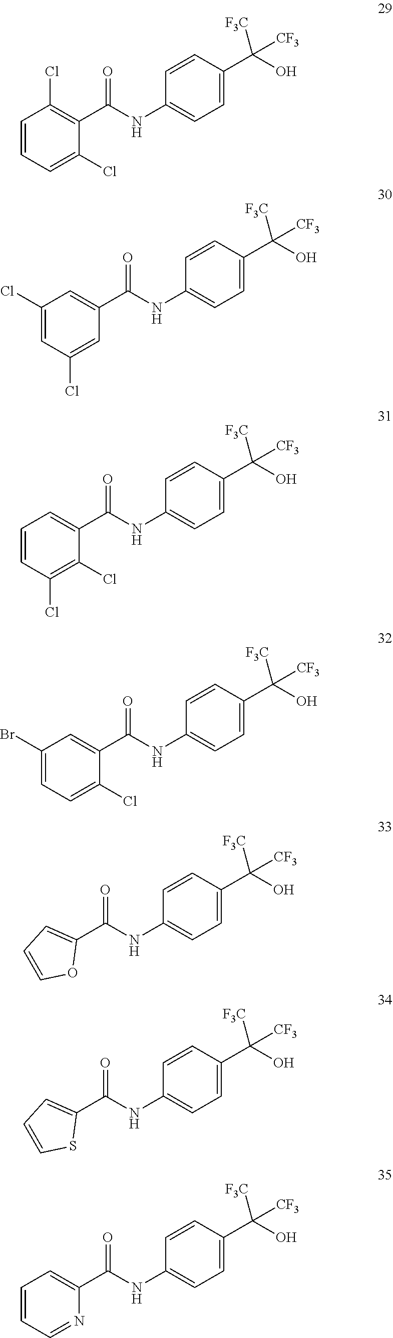 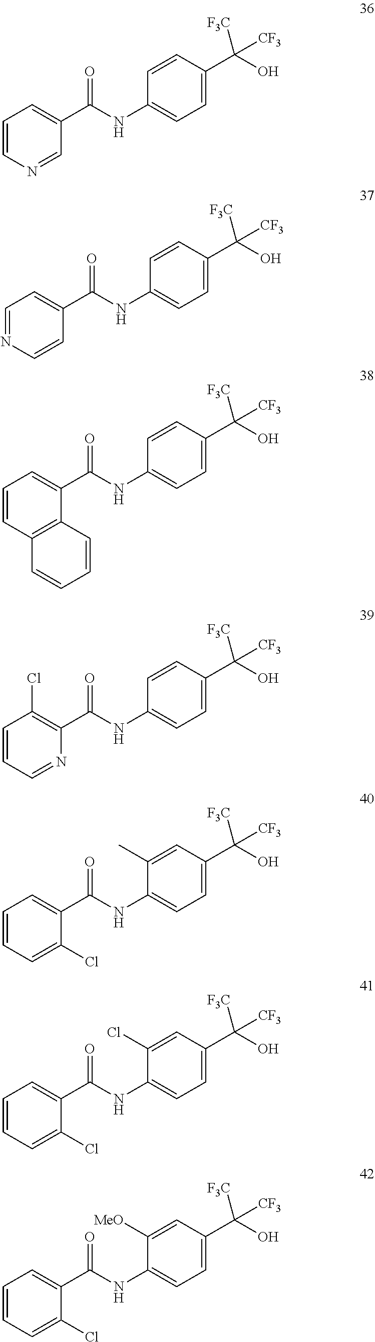 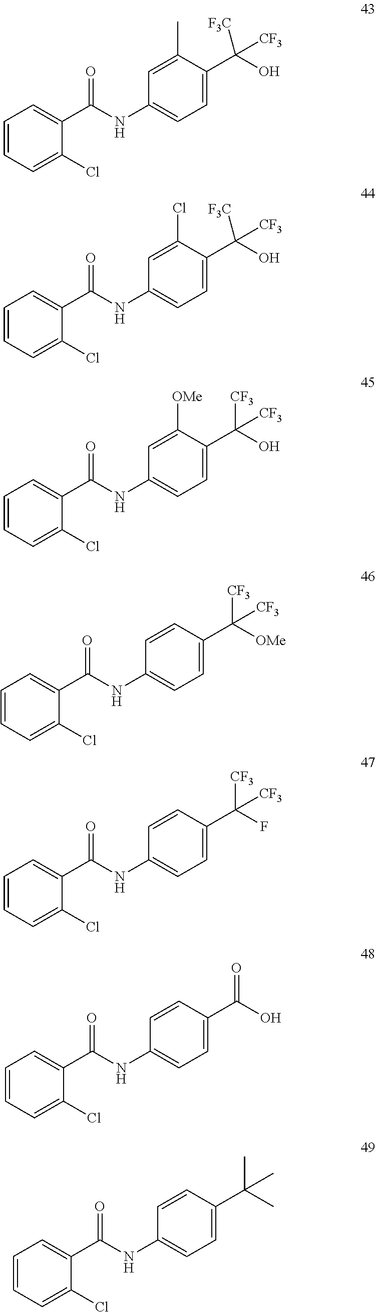 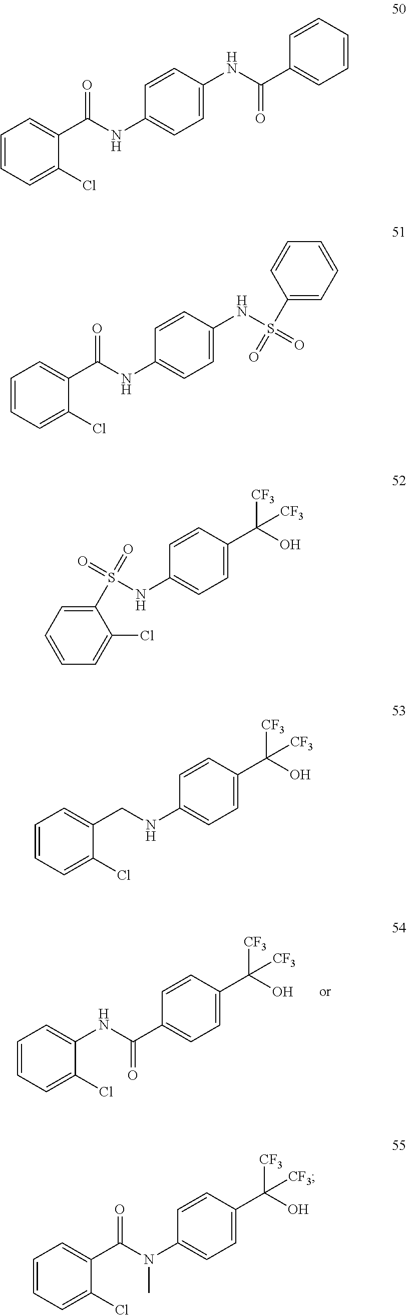 [SMILES: O=C(Nc1ccc(NC(=O)c2ccccc2Cl)cc1)c1ccccc1 ; O=C(Nc1ccc(NS(=O)(=O)c2ccccc2)cc1)c1ccccc1Cl ; O=S(=O)(Nc1ccc(C(O)(C(F)(F)F)C(F)(F)F)cc1)c1ccccc1Cl ; OC(c1ccc(NCc2ccccc2Cl)cc1)(C(F)(F)F)C(F)(F)F ; O=C(Nc1ccccc1Cl)c1ccc(C(O)(C(F)(F)F)C(F)(F)F)cc1 ; CN(C(=O)c1ccccc1Cl)c1ccc(C(O)(C(F)(F)F)C(F)(F)F)cc1]
or a pharmaceutically acceptable salt thereof.
